SEQUENCE LISTING (14 sequences)

SEQ ID NO: 1 - protein, 450 aa, Synthetic construct
QVQLVESGGGVVQPGRSLRLSCAASGFTFSNYGMHWVRQAPGEGLEWVAAIWFDASDKYYADAVKGRFTISRDNSKNTLYLQMNSLRAEDTAVYYCARDQAIFGVVPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPCVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 450 aa, Synthetic construct; identical to SEQ ID NO: 1

SEQ ID NO: 3 - protein, 31 aa, Synthetic construct
  MOD_RES 2: note = alpha-amino-isobutyric acid
HXEGTFTSDYSSYLEEQAAKEFIAWLVKGGG

SEQ ID NO: 4 - protein, 16 aa, Synthetic construct
GGGGSGGGGSGGGGSK

SEQ ID NO: 5 - protein, 5 aa, Synthetic construct
NYGMH

SEQ ID NO: 6 - protein, 17 aa, Synthetic construct
AIWFDASDKYYADAVKG

SEQ ID NO: 7 - protein, 11 aa, Synthetic construct
DQAIFGVVPDY

SEQ ID NO: 8 - protein, 11 aa, Synthetic construct
RASQSVSSNLA

SEQ ID NO: 9 - protein, 7 aa, Synthetic construct
GAATRAT

SEQ ID NO: 10 - protein, 9 aa, Synthetic construct
QQYNNWPLT

SEQ ID NO: 11 - protein, 466 aa, Homo sapiens
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 12 - protein, 430 aa, Homo sapiens
MTTSPILQLLLRLSLCGLLLQRAETGSKGQTAGELYQRWERYRRECQETLAAAEPPSVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 13 - protein, 493 aa, Homo sapiens
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVGRDPAAAPALWRRRGTAPPLSAIVSQVQSEIRRGWHHCRLRRSLGEEQRQLPERAFRALPSGSGPGEVPTSRGLSSGTLPGPGNEASRELESYC

SEQ ID NO: 14 - protein, 46 aa, Synthetic construct
  MOD_RES 2: note = alpha-amino-isobutyric acid
HXEGTFTSDYSSYLEEQAAKEFIAWLVKGGGGGGGSGGGGSGGGGS

(19) United States
(12) Patent Application Publication
(10) Pub. No.: US 2026/0166154 A1
(43) Pub. Date: Jun. 18, 2026

(54) METHODS FOR TREATING OBESITY

(71) Applicant: AMGEN INC., Thousand Oaks, CA (US)

(72) Inventors: Jennifer STRANDE, Thousand Oaks, CA (US); Murielle VENIANT-ELLISON, Thousand Oaks, CA (US); Rajneet OBEROI, Oak Park, CA (US)

(73) Assignee: AMGEN INC., Thousand Oaks, CA (US)

(21) Appl. No.: 19/127,648

(22) PCT Filed: Nov. 7, 2023
(86) PCT No.: PCT/US2023/078960

§ 371 Date: May 6, 2025

Related U.S. Application Data
(60) Provisional application No. 63/382,700, filed on Nov. 7, 2022.
(60) Provisional application No. 63/387,837, filed on Dec. 16, 2022.

(51) Int. Cl. A61K 47/26; A61K 9/00; A61K 47/10

(52) U.S. Cl. CPC A61K 47/26; A61K 9/0019; A61K 47/10

(57) ABSTRACT

The present invention relates to methods of treating obesity or type II diabetes using an antagonistic anti-GIPR antibody conjugated to a GLP-1R agonist. In particular, methods for treating obesity or type II diabetes in a patient in need thereof comprising administering to the patient an antagonistic anti-GIPR antibody conjugated to a GLP-1R agonist according to specific dosage regimens are disclosed. Pharmaceutical compositions and administration devices comprising an antagonistic anti-GIPR antibody conjugated to a GLP-1R agonist for use in the methods are also described.

### CROSS REFERENCE TO RELATED APPLICATIONS

This claims the benefit of U.S. Provisional Patent Application No. 63/382,700, filed Nov. 7, 2022, and U.S. Provisional Patent Application No. 63/387,837 filed Dec. 16, 2022, which are incorporated herein by reference in its entirety.

### SEQUENCE LISTING

The instant application contains a Sequence Listing which has been submitted electronically in XMLformat and is hereby incorporated by reference in its entirety. Said XML copy, created on Nov. 7, 2023, is named 10304-WO01-SEC_Seglisting.XML and is 16,012 bytes in size.

### FIELD OF THE INVENTION

The present invention relates to treatment of obesity using an antibody-peptide conjugate that selectively inhibits the glucose-dependent insulinotropic polypeptide receptor (GIPR) while agonizing glucagon-like peptide-1 receptor (GLP-1R).

### BACKGROUND OF THE INVENTION

As a multifactorial chronic disease, obesity has been an increasingly prevalent health problem worldwide (World Health Organ. 2016. Obesity and overweight: fact sheet 311). The obesity-associated comorbidities further add burden to the healthcare system (Apovian C M, 2016). The current unmet expectations of anti-obesity therapeutics support the need for developing safe and effective novel agents (Wright S M., Aronne L J. 2011; Valsamakis et al, 2017).

Glucose-dependent insulinotropic polypeptide (GIP) and glucagon-like peptide-1 (GLP-1) are gut-derived incretin hormones, well known for their ability to augment glucose stimulated insulin secretion. In addition to the incretin effects, GLP-1 promotes satiety via the GLP-1 receptor (GLP-1R) (Turton et al, 1996) while GIP promotes adiposity via the GIP receptor (GIPR) (Yip et al, 1998; Beck and Max, 1987; Hauner et al, 1988; Knapper et al, 1995). Several GLP-1 receptor agonists have been approved to treat type 2 diabetes and have demonstrated benefits in controlling obesity (Prasad-Reddy and Isaacs, 2015). Genome-wide association studies in human and mouse show that the GIPR locus contributes to body weight and GIPR knockout mice are protected from diet induced obesity (Berndt et al., 2013; Saxena et al., 2010; Speliotes et al., 2010: Althage et al., 2008; Miyawaki et al., 2002: Nasteska et al., 2014). Pharmacological inhibition of GIPR with anti-GIPR antibodies prevented body weight gain in diet-induced obese (DIO) mice and obese cynomolgus monkeys (Killion et al, 2018). In addition, GIPR antagonism in combination with GLP-IR agonism synergistically reduced body weight in DIO mice and obese cynomolgus monkeys (Killion et al, 2018), suggesting the potential for a GIPR/GLP-1R bispecific molecule for the treatment of obesity.

As a anti-GIPR/GLP-IR bispecific molecule, maridebart cafraglutide (also known as “AMG-133”) is engineered by conjugating a fully human monoclonal anti-human GIPR antagonist antibody and a GLP-1 analog agonist peptide using natural amino acid linkers. Here we report its discovery, preclinical development in DIO mice and obese cynomolgus monkeys, and clinical proof of concept study to evaluate the safety, tolerability, pharmacokinetics and pharmacodynamics of maridebart cafraglutide in patients with obesity.

### SUMMARY OF THE INVENTION

The present invention is based, in part, on the identification of a therapeutic regimen for effectively treating obesity or type II diabetes in patients. Accordingly, in one aspect, the present invention provides a method for treating obesity or type II diabetes in a patient in need thereof comprising administering to the patient a pharmaceutical composition comprising an antagonistic anti-GIPR antibody conjugated to a GLP-1R agonist at a dose of about 21 mg to about 840 mg every four weeks, a dose of about 21 mg to about 840 mg every six weeks, a dose of about 21 mg to about 840 mg every eight weeks, or a dose of about 21 mg to about 840 mg every twelve weeks.

In another aspect, the present invention is directed to use of an antagonistic anti-GIPR antibody conjugated to a GLP-1R agonist for preparation of a medicament for treating obesity or type II diabetes in a patient in need thereof, wherein the medicament is formulated for administration at a dose of about 21 mg to about 840 mg every four weeks, a dose of about 21 mg to about 840 mg every six weeks, a dose of about 21 mg to about 840 mg every eight weeks, or a dose of about 21 mg to about 840 mg every twelve weeks.

In certain embodiments, the dose is about 21 mg, about 70 mg, about 140 mg, about 280 mg, about 420 mg, about 560 mg, or about 840 mg every four weeks; about 21 mg, about 70 mg, about 140 mg, about 280 mg, about 420 mg, about 560 mg, or about 840 mg every six weeks; about 21 mg, about 70 mg, about 140 mg, about 280 mg, about 420 mg, about 560 mg, or about 840 mg every eight weeks, or about 21 mg, about 70 mg, about 140 mg, about 280 mg, about 420 mg, about 560 mg, or about 840 mg every twelve weeks.

In certain embodiments, the dose is about 280 mg every four weeks; about 280 mg every six weeks, about 280 mg every eight weeks, or about 280 mg every twelve weeks.

In certain embodiments, the dose is about 280 mg every four weeks.

In certain embodiments, the dose is about 420 mg every four weeks: about 420 mg every six weeks, about 420 mg every eight weeks, or about 420 mg every twelve weeks.

In certain embodiments, the pharmaceutical composition is administered once every four weeks.

In another aspect, the present invention provides a method for treating obesity or type II diabetes in a patient in need thereof comprising administering to the patient a pharmaceutical composition comprising an antagonistic anti-GIPR antibody conjugated to a GLP-1R agonist at a dose of about 21 mg to about 840 mg every month, a dose of about 21 mg to about 840 mg every one and a half months, a dose of about 21 mg to about 840 mg every two months, or a dose of about 21 mg to about 840 mg every three months.

In another aspect, the present invention is directed to use of an antagonistic anti-GIPR antibody conjugated to a GLP-1R agonist for preparation of a medicament for treating obesity or type II diabetes in a patient in need thereof, wherein the medicament is formulated for administration at a dose of about 21 mg to about 840 mg every month, a dose of about 21 mg to about 840 mg every one and a half months, a dose of about 21 mg to about 840 mg every two months, or a dose of about 21 mg to about 840 mg every three months.

In certain embodiments, the dose is about 21 mg, about 70 mg, about 140 mg, about 280 mg, about 420 mg, about 560 mg, or about 840 mg every month; about 21 mg, about 70 mg, about 140 mg, about 280 mg, about 420 mg, about 560 mg, or about 840 mg every one and a half months; about 21 mg, about 70 mg, about 140 mg, about 280 mg, about 420 mg, about 560 mg, or about 840 mg every two months, or about 21 mg, about 70 mg, about 140 mg, about 280 mg, about 420 mg, about 560 mg, or about 840 mg every three months.

In certain embodiments, the dose is about 280 mg every month; about 280 mg every one and a half months, about 280 mg every two months, or about 280 mg every three months.

In certain embodiments, the dose is about 280 mg every month.

In certain embodiments, the dose is about 420 mg every month; about 420 mg every one and a half months, about 420 mg every two months, or about 420 mg every three months.

In certain embodiments, the pharmaceutical composition is administered once per month.

In certain embodiments, the pharmaceutical composition is administered parenterally.

In certain embodiments, the parenteral administration is subcutaneous administration.

In certain embodiments, the pharmaceutical composition is administered to the patient with a syringe pre-filled with the pharmaceutical composition.

In certain embodiments, the pharmaceutical composition is administered to the patient with an autoinjector.

In certain embodiments, administration of the pharmaceutical composition does not substantially cause an adverse side effect in the patient.

In certain embodiments, the patient has a body mass index (“BMI”) greater than 30 kg/m2, 35 kg/m2, or 40 kg/m2.

In certain embodiments, the patient has HbA1c≥7% and ≤10% (53 to 86 mmol/mol).

In certain embodiments, the patient has been treated with metformin, a sulfonylurea, or a sodium-glucose cotransporter 2 (SGLT2) inhibitor as monotherapy or combination therapy.

In certain embodiments, the patient has or is diagnosed with type II diabetes. In other embodiments, the patient is obese, but does not have type II diabetes.

In certain embodiments, the patient has not previously received therapy for obesity.

In certain embodiments, the anti-GIPR antibody comprises a CDRH1 comprising the amino acid sequence of SEQ ID NO:5, a CDRH2 comprising the amino acid sequence of SEQ ID NO:6, a CDRH3 comprising the amino acid sequence of SEQ ID NO:7, a CDRL1 comprising the amino acid sequence of SEQ ID NO:8, a CDRL2 comprising the amino acid sequence of SEQ ID NO:9, and a CDRL3 comprising the amino acid sequence of SEQ ID NO: 10.

In certain embodiments, the antagonistic anti-GIPR antibody comprises a heavy chain comprising the amino acid sequence of SEQ ID NO:1, and a light chain comprising the amino acid sequence of SEQ ID NO: 2.

In certain embodiments, the GLP-IR agonist comprises the amino acid sequence of SEQ ID NO:3.

In certain embodiments, the antagonistic anti-GIPR antibody is linked to the GLP-1R agonist via a peptide linker comprising SEQ ID NO: 4.

In one embodiment, the antagonistic anti-GIPR antibody conjugated to a GLP-1R agonist is maridebart cafraglutide.

In certain embodiments, the pharmaceutical composition further comprises a buffer.

In certain embodiments, the buffer is an acetate buffer.

In certain embodiments, the pharmaceutical composition further comprises a surfactant.

In certain embodiments, the surfactant is polysorbate 20 or polysorbate 80.

In certain embodiments, the pharmaceutical composition further comprises a stabilizing agent.

In certain embodiments, the stabilizing agent is sucrose.

### BRIEF DESCRIPTION OF THE DRAWINGS

FIG. 1 depicts Physical parameters after single and multiple doses of AMG 133.

FIG. 2 depicts Table 1A: Baseline Characteristics for treatment groups in subjects receiving single ascending doses of AMG 133 or Placebo

FIG. 3 depicts Changes in metabolic and inflammatory parameters in subjects in a multiple ascending dose study with AMG 133.

FIG. 4 depicts Table 1B: Baseline Characteristics for treatment groups in subjects receiving multiple ascending doses of AMG 133 or Placebo

FIG. 5 depicts Table 3A: Treatment-emergent adverse events after a single dose for placebo and AMG 133

FIG. 6 depicts Table 3B: Treatment-emergent adverse events after a multiple doses for placebo and AMG 133

FIG. 7 depicts Table 4A: Vital sign measures in subjects after a single dose for placebo or AMG 133

FIG. 8 depicts Table 4B: Vital sign measures in subjects after multiple doses for placebo or AMG 133.

FIG. 9 depicts the Phase 2 Study Schema (Cohort A)—Part 1 (Subjects without Type 1 or 2 Diabetes Mellitus).

FIG. 10 depicts the Phase 2 Study Schema (Cohort B)—Part 1 (Subjects with Diabetes Mellitus).

FIG. 11 depicts the Phase 2 Study Schema—Part 2.

FIG. 12 depicts the Phase 2 Investigational Products.

FIG. 13A depicts the Phase 2 Part 1 Study Groups for Cohort A (Subjects without Type 1 or 2 Diabetes Mellitus).

FIG. 13BA depicts the Phase 2 Part 1 Study Groups for Cohort A (Subjects with Diagnosis of Type 2 Diabetes Mellitus).

FIG. 14 depicts Phase 2 Part 2 Study Groups.

### DETAILED DESCRIPTION

The present disclosure provides a method of treating a metabolic disorder, such as a disorder of glucose metabolism (e.g. Type 2 diabetes, elevated glucose levels, elevated insulin levels, dyslipidemia, metabolic syndrome (Syndrome X or insulin resistance syndrome), glucosuria, metabolic acidosis, diabetic neuropathy, diabetic nephropathy, diabetic retinopathy, diabetic cardiomyopathy, Type 1 diabetes, obesity and conditions exacerbated by obesity) by blocking or interfering with the biological activity of GIP. In one embodiment, a therapeutically effective amount of an isolated human GIPR binding protein conjugated to a GLP-1 receptor agonist is administered to a subject in need thereof. Methods of administration and delivery are also provided.

In one embodiment, the human GIPR has a sequence comprising a sequence selected from the group consisting of SEQ ID NO: 11, SEQ ID NO: 12, and SEQ ID NO: 13.

The 466 amino acid sequence of human GIPR is (Volz et al., FEBS Lett. 373:23-29 (1995); NCBI Reference Sequence: NP_0001555):

(SEQ ID NO: 11)
MTTSPILQLL LRLSLCGLLL QRAETGSKGQ TAGELYQRWE
RYRRECQETL AAAEPPSGLA CNGSFDMYVC WDYAAPNATA
RASCPWYLPW HHHVAAGFVL RQCGSDGQWG LWRDHTQCEN
PEKNEAFLDQ RLILERLQVM YTVGYSLSLA TLLLALLILS
LFRRLHCTRN YIHINLFTSF MLRAAAILSR DRLLPRPGPY
LGDQALALWN QALAACRTAQ IVTQYCVGAN YTWLLVEGVY
LHSLLVLVGG SEEGHFRYYL LLGWGAPALF VIPWVIVRYL
YENTQCWERN EVKAIWWIIR TPILMTILIN FLIFIRILGI
LLSKLRTRQM RCRDYRLRLA RSTLTLVPLL GVHEVVFAPV
TEEQARGALR FAKLGFEIFL SSFQGFLVSV LYCFINKEVQ
SEIRRGWHHC RLRRSLGEEQ RQLPERAFRA LPSGSGPGEV
PTSRGLSSGT LPGPGNEASR ELESYC

A 430 amino acid isoform of human GIPR (isoform X1), predicted by automated computational analysis, has the sequence (NCBI Reference Sequence XP_005258790):

(SEQ ID NO: 12)
MITSPILQLL LRLSLCGLLL QRAETGSKGQ TAGELYQRWE
RYRRECQETL AAAEPPSVAA GFVLRQCGSD GQWGLWRDHT
QCENPEKNEA FLDQRLILER LQVMYTVGYS LSLATLLLAL
LILSLFRRLH CTRNYIHINL FTSFMLRAAA ILSRDRLLPR
PGPYLGDQAL ALWNQALAAC RTAQIVTQYC VGANYTWLLV
EGVYLHSLLV LVGGSEEGHF RYYLLLGWGA PALFVIPWVI
VRYLYENTQC WERNEVKAIW WIIRTPILMT ILINFLIFIR
ILGILLSKLR TRQMRCRDYR LRLARSTLTL VPLLGVHEVV
FAPVTEEQAR GALRFAKLGF EIFLSSFQGF LVSVLYCFIN
KEVQSEIRRG WHHCRLRRSL GEEQRQLPER AFRALPSGSG
PGEVPTSRGL SSGTLPGPGN EASRELESYC

A 493 amino acid isoform of human GIPR, produced by alternative splicing, has the sequence (Gremlich et al., Diabetes 44:1202-8 (1995); UniProtKB Sequence Identifier: P48546-2):

(SEQ ID NO: 13)
MTTSPILQLL LRLSLCGLLL QRAETGSKGQ TAGELYQRWE
RYRRECQETL AAAEPPSGLA CNGSFDMYVC WDYAAPNATA
RASCPWYLPW HHHVAAGFVL RQCGSDGQWG LWRDHTQCEN
PEKNEAFLDQ RLILERLQVM YTVGYSLSLA TLLLALLILS
LFRRLHCTRN YIHINLFTSF MLRAAAILSR DRLLPRPGPY
LGDQALALWN QALAACRTAQ IVTQYCVGAN YTWLLVEGVY
LHSLLVLVGG SEEGHFRYYL LLGWGAPALF VIPWVIVRYL
YENTQCWERN EVKAIWWIIR TPILMTILIN FLIFIRILGI
LLSKLRTRQM RCRDYRLRLA RSTLTLVPLL GVHEVVFAPV
TEEQARGALR FAKLGFEIFL SSFQGFLVSV LYCFINKEVG
RDPAAAPALW RRRGTAPPLS AIVSQVQSEI RRGWHHCRLR
RSLGEEQRQL PERAFRALPS GSGPGEVPTS RGLSSGTLPG
PGNEASRELE SYC

Recombinant polypeptide and nucleic acid methods used herein, including in the Examples, are generally those set forth in Sambrook et al., Molecular Cloning: A Laboratory Manual (Cold Spring Harbor Laboratory Press, 1989) or Current Protocols in Molecular Biology (Ausubel et al., eds., Green Publishers Inc. and Wiley and Sons 1994), both of which are incorporated herein by reference for any purpose.

The section headings used herein are for organizational purposes only and are not to be construed as limiting the subject matter described.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present application are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press. Cold Spring Harbor, N.Y. (2001), Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

It should be understood that this invention is not limited to the particular methodology, protocols, and reagents, etc., described herein and as such may vary. The terminology used herein is for the purpose of describing particular embodiments only, and is not intended to limit the scope of the disclosed, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

Following convention, as used herein “a” and “an” mean “one or more” unless specifically indicated otherwise.

As used herein, the terms “amino acid” and “residue” are interchangeable and, when used in the context of a peptide or polypeptide, refer to both naturally occurring and synthetic amino acids, as well as amino acid analogs, amino acid mimetics and non-naturally occurring amino acids that are chemically similar to the naturally occurring amino acids.

A “naturally occurring amino acid” is an amino acid that is encoded by the genetic code, as well as those amino acids that are encoded by the genetic code that are modified after synthesis, e.g., hydroxyproline, γ-carboxyglutamate, and O-phosphoserine. An amino acid analog is a compound that has the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but will retain the same basic chemical structure as a naturally occurring amino acid.

An “amino acid mimetic” is a chemical compound that has a structure that is different from the general chemical structure of an amino acid, but that functions in a manner similar to a naturally occurring amino acid. Examples include a methacryloyl or acryloyl derivative of an amide, β-, γ-, δ-imino acids (such as piperidine-4-carboxylic acid) and the like.

A “non-naturally occurring amino acid” is a compound that has the same basic chemical structure as a naturally occurring amino acid, but is not incorporated into a growing polypeptide chain by the translation complex. “Non-naturally occurring amino acid” also includes, but is not limited to, amino acids that occur by modification (e.g., posttranslational modifications) of a naturally encoded amino acid (including but not limited to, the 20 common amino acids) but are not themselves naturally incorporated into a growing polypeptide chain by the translation complex. A non-limiting lists of examples of non-naturally occurring amino acids that can be inserted into a polypeptide sequence or substituted for a wild-type residue in polypeptide sequence include p-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα-methylhomocitrulline (Nα-MeHoCit), ornithine (Orn), Nα-Methylomithine (Nα-MeOrn or NMeOrn), sarcosine (Sar), homolysine (hLys or hK), homoarginine (hArg or hR), homoglutamine (hQ), Nα-methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2-Nal), 1,2,3,4-tetrahydroisoquinoline (Tic), 2-indanylglycine (IgI), para-iodophenylalanine (pI-Phe), para-aminophenylalanine (4AmP or 4-Amino-Phe), 4-guanidino phenylalanine (Guf), glycyllysine (abbreviated “K(Ne-glycyl)” or “K(glycyl)” or “K(gly)”), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), a-aminoadipic acid (Aad). Nα-methyl valine (NMeVal). N-α-methyl leucine (NMeLeu), Nα-methylnorleucine (NMeNle), cyclopentylglycine (Cpg), cyclohexylglycine (Chg), acetylarginine (acetylarg), α, β-diaminopropionoic acid (Dpr), α, γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aninopropionic acid, piperidinic acid, aminocaprioic acid, amninoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methylglycine, N-methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of those specifically listed.

The term “isolated nucleic acid molecule” refers to a single or double-stranded polymer of deoxyribonucleotide or ribonucleotide bases read from the 5′ to the 3′ end (e.g., a GIPR nucleic acid sequence provided herein), or an analog thereof, that has been separated from at least about 50 percent of polypeptides, peptides, lipids, carbohydrates, polynucleotides or other materials with which the nucleic acid is naturally found when total nucleic acid is isolated from the source cells. Preferably, an isolated nucleic acid molecule is substantially free from any other contaminating nucleic acid molecules or other molecules that are found in the natural environment of the nucleic acid that would interfere with its use in polypeptide production or its therapeutic, diagnostic, prophylactic or research use.

The term “isolated polypeptide” refers to a polypeptide (e.g., a GIPR polypeptide sequence provided herein or an antigen binding protein of the present invention) that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

A composition of the present invention that includes a GLP-1 receptor agonist of the invention covalently linked, attached, or bound, either directly or indirectly through a linker moiety, to another an antagonistic anti-GIPR antibody of the invention or is a “conjugate” or “conjugated” molecule, whether conjugated by chemical means (e.g., post-translationally or post-synthetically).

The term “encoding” refers to a polynucleotide sequence encoding one or more amino acids. The term does not require a start or stop codon.

In certain embodiments, the present invention is directed to a method for treating obesity or type II diabetes in a patient in need thereof comprising administering to the patient a pharmaceutical composition comprising an antagonistic anti-GIPR antibody conjugated to a GLP-1R agonist at a dose of about 21 mg to about 840 mg every four weeks, a dose of about 21 mg to about 840 mg every six weeks, or a dose of about 21 mg to about 840 mg every eight weeks. In another aspect, the present invention is directed to use of an antagonistic anti-GIPR antibody conjugated to a GLP-IR agonist for preparation of a medicament for treating obesity or type II diabetes in a patient in need thereof, wherein the medicament is formulated for administration at a dose of about 21 mg to about 840 mg every four weeks, a dose of about 21 mg to about 840 mg every six weeks, or a dose of about 21 mg to about 840 mg every eight weeks. In certain embodiments, the dose is about 21 mg, about 70 mg, about 140 mg, about 280 mg, about 560 mg, or about 840 mg every four weeks; about 21 mg, about 70 mg, about 140 mg, about 280 mg, about 560 mg, or about 840 mg every six weeks: or about 21 mg, about 70 mg, about 140 mg, about 280 mg, about 560 mg, or about 840 mg every eight weeks. In certain embodiments, the dose is about 280 mg every four weeks; about 280 mg every six weeks, or about 280 mg every eight weeks.

The weekly and monthly dosing schedules of the antagonistic anti-GIPR antibody conjugated to a GLP-1R agonist is similar among patients regardless of body weight. In other words, in these embodiments, the dosage of antagonistic anti-GIPR antibody conjugated to a GLP-1R agonist is a total dose and is not adjusted for a patient's body weight. In one embodiment, an antagonistic anti-GIPR antibody conjugated to a GLP-1R agonist is administered to the patient at a total dose of about 280 mg every month.

In certain embodiments, the monthly dose of antagonistic anti-GIPR antibody conjugated to a GLP-1R agonist may be based upon a patient's body weight. For example, in some embodiments, the monthly dose of an antagonistic anti-GIPR antibody conjugated to a GLP-1R agonist may range from about 0.3 mg/kg to about 3.5 mg/kg of body weight, from about 0.5 mg/kg to about 3 mg/kg of body weight, or from about 1 mg/kg to about 2.5 mg/kg of body weight. For instance, the monthly dose of antagonistic anti-GIPR antibody conjugated to a GLP-1R agonist may be about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.2 mg/kg, about 3.3 mg/kg, about 3.4 mg/kg, or about 3.5 mg/kg of body weight. In one embodiment, the monthly dose of antagonistic anti-GIPR antibody conjugated to a GLP-1R agonist is about 0.8 mg/kg to about 1.2 mg/kg of body weight. In another embodiment, the monthly dose of antagonistic anti-GIPR antibody conjugated to a GLP-1R agonist is about 1.6 mg/kg to about 2.2 mg/kg of weight.

The dose of antagonistic anti-GIPR antibody conjugated to a GLP-1R agonist can be administered in a single administration or divided among multiple administrations over the course of the dosing frequency period. For example, in certain embodiments, the therapeutically effective dose of antagonistic anti-GIPR antibody conjugated to a GLP-1R agonist is administered in a single administration each frequency period. Thus, in some embodiments, any of the doses of antagonistic anti-GIPR antibody conjugated to a GLP-1R agonist described herein can be administered to the patient once a month (QM dosing). Patients on a QM dosing regimen are typically administered the antagonistic anti-GIPR antibody conjugated to a GLP-1R agonist every 24 to 36 days, preferably, every 28 to 35 days, more preferably, every 28 to 31 days, or even more preferably, every 28 days or every 30 days. In these and other embodiments, the monthly dose is administered to the patient as a bolus injection, for example, using a self-injection device as described herein. For instance, a monthly dose of 280 mg can be administered to the patient as a single bolus injection of 280 mg optionally with an autoinjector, pen injector, or pre-filled syringe containing the 280 mg dose. In certain embodiments, the monthly dose is given in two or more consecutive injections. By way of example, a monthly dose of 280 mg can be administered to the patient in two consecutive injections of 140 mg optionally with two injection devices (e.g. autoinjectors, pen injectors, or pre-filled syringes) containing a 140 mg dose.

In alternative embodiments, the doses of antagonistic anti-GIPR antibody conjugated to a GLP-1R agonist are divided among two or more administrations over the course of the dosing frequency period. For example, for a dosing frequency period of one month, the monthly dose may be divided into four doses and administered on a weekly basis or divided into two doses and administered every two weeks. Any of the doses of the antagonistic anti-GIPR antibody conjugated to a GLP-1R agonist described herein can be divided among two or more administrations. The number of administrations and intervening interval can be adjusted for a particular patient depending on the severity of obesity, the age of the patient, the physical health of the patient, concomitant treatment with other medications, and/or the presence of other conditions.

In certain embodiments, the dosing frequency period for the doses of an antagonistic anti-GIPR antibody conjugated to a GLP-IR agonist that are described herein is monthly. In other words, the dosages of antagonistic anti-GIPR antibodies conjugated to a GLP-1R agonist are monthly dosages (monthly dosages are equal to one every four weeks), but can be administered in a single administration (i.e. once a month; QM dosing) or divided among multiple administrations over the course of the month (e.g. ½ the monthly dose administered every two weeks).

In certain embodiments, the patient has a body mass index (“BMI”) greater than 30 kg/m2, 35 kg/m2 or 40 kg/m2. In certain embodiments, the patient has a BMI greater than 30 kg/m2 and less than 40 kg/m2. In certain embodiments, the patient has a BMI greater than 35 kg/m2 and less than 40 kg/m2. In certain embodiments, the patient has a BMI greater than 30 kg/m2 and less than 35 kg/m2. The measurements can also be converted to lbs/in².

Body Mass Index (BMI) is a person's weight in kilograms (or pounds) divided by the square of height in meters (or feet). A high BMI can indicate high body fatness.

In certain embodiments, treating obesity comprises promoting weight loss in a patient. Accordingly, the present invention is directed to a method of treating obesity wherein the patient loses about 5% of body weight in 52 weeks, about 10% of body weight in 52 weeks, about 15% of body weight in 52 weeks, about 20% of body weight in 52 weeks, about 25% of body weight in 52 weeks, or about 30% of body weight in 52 weeks. “Body weight” means a patient's weight before the first administration of the pharmaceutical composition.

In certain embodiments, treating obesity comprises maintaining body weight by administering a maintenance dose to patient. Accordingly, the present invention is directed to a method of treating obesity wherein the patient's body does not fluctuate more than ±0.5% of goal body weight. ±1.0% of goal body weight, ±1.5% of goal body weight, ±2.0% of goal body weight, ±2.5% of goal body weight, ±3.0% of goal body weight, ±3.5% of goal body weight, ±4.0% of goal body weight, ±4.5% of goal body weight, or ±5.0% of goal body weight. “Goal body weight” means the weight a patient means to maintain. A maintenance dose can be the same amount and frequency as a dose that promotes weight loss. Alternatively, a maintenance dose can be 1) lower amount than a dose that promotes weight loss, 2) administered less frequently than a dose that promotes weight loss, or 3) both a lower amount than a dose that promotes weight loss and administered less frequently than a dose that promotes weight loss.

In certain embodiments, the patient has been diagnosed with Type II diabetes. In certain embodiments, the patient has HbA1c≥7% and ≤10% (53 to 86 mmol/mol).

In certain embodiments, the patient has been treated with metformin, a sulfonylurea, or a sodium-glucose cotransporter 2 (SGLT2) inhibitor as monotherapy or combination therapy.

In some embodiments of the methods of the invention, the antagonistic anti-GIPR antibody conjugated to a GLP-IR agonist is administered to the patient over the course of a set treatment period. A “treatment period” begins upon administration of a first dose of antagonistic anti-GIPR antibody conjugated to a GLP-1R agonist and ends upon administration of a final dose of anti-GIPR antibody or binding fragment. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more.

Administration of an antagonistic anti-GIPR antibody conjugated to a GLP-1R agonist according to the methods of the invention preferably causes few or no adverse side effects in the patient. As used herein, the term “adverse side effect” refers to any abnormality, defect, mutation, lesion, degeneration, harmful or undesirable reaction, symptom, or injury, which may be caused by taking the drug. In some embodiments, administration of the antagonistic anti-GIPR antibody conjugated to a GLP-1R agonist does not substantially cause one or more adverse side effects associated with other obesity treatments. Side effects associated with other weight loss treatments include, but are not limited to, fatigue, nausea, dizziness, insomnia, depression, reduced exercise tolerance, tremor, paresthesia, teratogenicity, and cognitive difficulty. In other embodiments, administration of the antagonistic anti-GIPR antibody conjugated to a GLP-1R agonist is associated with a lower rate or number of adverse side effects as compared to the rate or number of adverse side effects associated with other obesity treatments. In yet other embodiments, administration of the antagonistic anti-GIPR antibody conjugated to a GLP-1R agonist is associated with a lower rate of discontinuation due to adverse side effects as compared to the rate of discontinuation due to adverse side effects associated with other weight loss treatments. In certain embodiments, the number and type of adverse side effects associated with administration of the antagonistic anti-GIPR antibody conjugated to a GLP-1R agonist is not statistically different than the number and type of adverse side effects associated with administration of placebo.

The methods described herein comprise administering to a patient an antagonistic anti-GIPR antibody conjugated to a GLP-1R agonist thereof. The term “antibody,” as used herein, refers to an intact immunoglobulin of any isotype, that can compete with the intact antibody for specific binding to the target antigen, and includes, for instance, chimeric, humanized, or fully human. The structural units of antibodies typically comprise one or more tetramers, each composed of two identical couplets of polypeptide chains, though some species of mammals also produce antibodies having only a single heavy chain. In a typical antibody, each pair or couplet includes one full-length “light” chain (in certain embodiments, about 25 kDa) and one full-length “heavy” chain (in certain embodiments, about 50-70 kDa). Each individual immunoglobulin chain is composed of several “immunoglobulin domains.” each consisting of roughly 90 to 110 amino acids and expressing a characteristic folding pattern. These domains are the basic units of which antibody polypeptides are composed. The amino-terminal portion of each chain typically includes a variable domain that is responsible for antigen recognition. The carboxy-terminal portion is more conserved evolutionarily than the other end of the chain and is referred to as the “constant region” or “C region.” Human light chains generally are classified as kappa and lambda light chains, and each of these contains one variable domain and one constant domain. Heavy chains are typically classified as mu, delta, gamma, alpha, or epsilon chains, and these define the antibody's isotype as IgM, IgD, IgG, IgA, and IgE, respectively. IgG has several subtypes, including, but not limited to, IgG1, IgG2, IgG3, and IgG4. IgM subtypes include IgM, and IgM2. IgA subtypes include IgA1 and IgA2. In humans, the IgA and IgD isotypes contain four heavy chains and four light chains: the IgG and IgE isotypes contain two heavy chains and two light chains; and the IgM isotype contains five heavy chains and five light chains. The heavy chain C region typically comprises one or more domains that may be responsible for effector function. The number of heavy chain constant region domains will depend on the isotype. IgG heavy chains, for example, each contains three C region domains known as CH1, CH2 and CH3. The antibodies that can be employed in the methods of the invention can have any of these isotypes and subtypes. In certain embodiments, the anti-GIPR antibody is of the IgG1, IgG2, or IgG4 subtype. In one particular embodiment, the anti-GIPR antibody is an IgG2 antibody (e.g. comprises a human IgG2 constant domain). In another particular embodiment, the anti-GIPR antibody is an IgG1 antibody (e.g. comprises a human IgG1 constant domain).

In full-length light and heavy chains, the variable and constant regions are joined by a “J” region of about twelve or more amino acids, with the heavy chain also including a “D” region of about ten more amino acids. See, e.g., Fundamental Immunology, 2nd ed., Ch. 7 (Paul. W., ed.) 1989, New York: Raven Press (hereby incorporated by reference in its entirety for all purposes). The variable regions of each light/heavy chain pair typically form the antigen binding site. Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., GIPR). From N-terminal to C-terminal, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991. NIH, Bethesda, MD), or Chothia & Lesk, 1987, J. Mol. Biol. 196:901-917; Chothia et al., 1989. Nature 342:878-883.

The term “binding fragment” is used interchangeably herein with the term “antigen-binding fragment” and refers to a portion (regardless of how that portion is obtained or synthesized) of an antibody that lacks at least some of the amino acids present in a full-length heavy chain and/or light chain, but which is capable of specifically binding to an antigen. Such fragments are biologically active in that they bind specifically to the target antigen and can compete with other antigen binding proteins, including intact antibodies, for specific binding to a given epitope. In one aspect, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of antigen binding proteins, including intact antibodies. Immunologically functional immunoglobulin fragments include, but are not limited to, Fab, Fab′, F(ab′)₂, Fv, domain antibodies and single-chain antibodies, and may be derived from any mammalian source, including but not limited to human, mouse, rat, camelid or rabbit.

An antibody binding fragment may be a synthetic or genetically engineered protein. For example, antibody binding fragments include isolated fragments consisting of the light chain variable region, “Fv” fragments consisting of the variable regions of the heavy and light chains, and recombinant single chain polypeptide molecules in which light and heavy variable regions are connected by a peptide linker (scFv proteins). Another form of an antibody binding fragment is a peptide comprising one or more complementarity determining regions (CDRs) of an antibody. CDRs (also termed “minimal recognition units” or “hypervariable region”) are obtained by, e.g., constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

An antibody or binding fragment is said to “specifically bind” to its target antigen when the dissociation constant (K_{D}) is ≤10⁻⁶ M. The antibody or binding fragment specifically binds the target antigen with “high affinity” when the K_{D} is ≤1×10⁻⁸ M. In one embodiment, the antibodies bind to human GIPR with a K_{D}≤5×10⁻⁷ M. In another embodiment, the antibodies or binding fragments bind to human GIPR with a K_{D}≤1×10⁻⁷ M. In still another embodiment, the antibodies or binding fragments bind to human GIPR with a K_{D}≤5×10⁻⁸ M. In another embodiment, the antibodies or binding fragments bind to human GIPR with a K_{D}≤1×10⁻⁸ M. In another embodiment the antibodies or binding fragments bind to human GIPR with a K_{D}≤5×10⁻⁹ M. In certain embodiments the antibodies or binding fragments bind to human GIPR with a K_{D}≤1×10⁻⁹ M. In other embodiments, the antibodies or binding fragments bind to human GIPR with a K_{D}≤5×10⁻¹⁰ M. In still other embodiments, the antibodies or binding fragments bind to human GIPR with a K_{D}≤1×10⁻¹⁰ M. Affinity is determined using a variety of techniques, an example of which is an affinity ELISA assay. In various embodiments, affinity is determined by a BIAcore assay. In some embodiments, affinity is determined by a kinetic method. In other embodiments, affinity is determined by an equilibrium/solution method. In certain embodiments, affinity is determined by a FACS binding assay. WO 2010/075238, which is hereby incorporated by reference in its entirety, describes suitable affinity assays for determining the affinity for anti-GIPR antibodies.

An antibody or antigen-binding fragment thereof “selectively inhibits” a specific receptor relative to other receptors when the IC50 of the antibody or antigen-binding fragment thereof in an inhibition assay of the specific receptor is at least 50-fold lower than the IC50 in an inhibition assay of another “reference” receptor. An “IC50” is the amount of a drug or substance that is needed to inhibit a given biological process by half. The IC50 of any particular substance or antagonist can be determined by constructing a dose-response curve and examining the effect of different concentrations of the drug or antagonist on reversing agonist activity in a particular functional assay. IC50 values can be calculated for a given antagonist or drug by determining the concentration needed to inhibit half of the maximum biological response of the agonist. Thus, the IC50 value for any anti-GIPR antibody or binding fragment can be calculated by determining the concentration of the antibody or binding fragment needed to inhibit half of the maximum biological response of the GIP ligand in activating the GIPR in any functional assay.

The anti-GIPR antibodies and binding fragments thereof for use in the methods disclosed herein may comprise one heavy chain CDR1 (“CDRH1”), and/or one heavy chain CDR2 (“CDRH2”), and/or one heavy chain CDR3 (“CDRH3”), and/or one light chain CDR1 (“CDRL1”), and/or one light chain CDR2 (“CDRL2”), and/or one light chain CDR3 (“CDRL3”). In some embodiments, the antagonistic anti-GIPR antibody conjugated to a GLP-IR agonist comprises at least one heavy chain variable region comprising a CDRH1. CDRH2, and CDRH3 and at least one light chain variable region comprising a CDRL1, CDRL2, and CDRL3.

Complementarity determining regions (CDRs) and framework regions (FR) of a given antibody may be identified using the system described by Kabat et al. in Sequences of Proteins of Immunological Interest, 5th Ed.. US Dept. of Health and Human Services, PHS. NIH. NIH Publication no. 91-3242, 1991.

In certain embodiments, the anti-GIPR antibody comprises a CDRH1 comprising the amino acid sequence of SEQ ID NO:5, a CDRH2 comprising the amino acid sequence of SEQ ID NO:6, a CDRH3 comprising the amino acid sequence of SEQ ID NO:7, a CDRL1 comprising the amino acid sequence of SEQ ID NO:8, a CDRL2 comprising the amino acid sequence of SEQ ID NO:9, and a CDRL3 comprising the amino acid sequence of SEQ ID NO: 10.

SEQ ID NO: 5
NYGMH
SEQ ID NO: 6
AIWFDASDKYYADAVKG
SEQ ID NO: 7
DQAIFGVVPDY
SEQ ID NO: 8
RASQSVSSNLA
SEQ ID NO: 9
GAATRAT
SEQ ID NO: 10
QQYNNWPLT

In certain embodiments, the antagonistic anti-GIPR antibody comprises a heavy chain comprising the amino acid sequence of SEQ ID NO:1, and a light chain comprising the amino acid sequence of SEQ ID NO:2.

SEQ ID NO: 1
QVQLVESGGG VVQPGRSLRL SCAASGFTFS NYGMHWVRQA
PGEGLEWVAA IWFDASDKYY ADAVKGRFTI SRDNSKNTLY LQMNSLRAED TAVYYCARDQ
AIFGVVPDYW GQGTLVTVSS ASTKGPSVFP LAPSSKSTSG GTAALGCLVK DYFPEPVTVS
WNSGALTSGV HTFPAVLQSS GLYSLSSVVT VPSSSLGTQT YICNVNHKPS NTKVDKKVEP
KSCDKTHTCP PCPAPELLGG PSVFLFPPKP KDTLMISRTP EVTCVVVDVS HEDPCVKFNW
YVDGVEVHNA KTKPCEEQYG STYRCVSVLT VLHQDWLNGK EYKCKVSNKA LPAPIEKTIS
KAKGQPREPQ VYTLPPSREE MTKNQVSLTC LVKGFYPSDI AVEWESNGQP ENNYKTTPPV
LDSDGSFFLY SKLTVDKSRW QQGNVFSCSV MHEALHNHYT QKSLSLSPGK
SEQ ID NO: 2
EIVMTQSPAT LSVSPGERAT LSCRASQSVS SNLAWYQQKP
GQAPRLLIYG AATRATGIPA RVSGSGSGTE FTLTISSLQS EDFAVYYCQQ YNNWPLTFGG
GTKVEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC

In certain embodiments, the GLP-1R agonist comprises the amino acid sequence of SEQ ID NO:3.

SEQ ID NO: 3
H[Aib]EGTFTSDYSSYLEEQAAKEFIAWLVKGGG

In certain embodiments, the antagonistic anti-GIPR antibody is linked to the GLP-1R agonist via a peptide linker comprising SEQ ID NO: 4.

SEQ ID NO: 4
GGGGSGGGGSGGGGSK

Maridebart cafraglutide (also known as “AMG-133”, “AMG133”, and “AMG 133”) is engineered by conjugating a fully human monoclonal anti-human GIPR antagonist antibody and a GLP-1 analog agonist peptide using natural amino acid linkers. The heavy chain of AMG 133 consists of SEQ ID NO: 1. The light chain of AMG 133 consists of SEQ ID NO: 2. AMG 133 comprises a linker (SEQ ID NO:4) bound to cysteine 275 of SEQ ID NO:1 via the C-terminal lysine of SEQ ID NO: 4. AMG 133 further comprises the C-terminal of SEQ ID NO: 3 bound to the N-terminal of SEQ ID NO: 4. Accordingly, the sequence C-terminal lysine of the peptide H[Aib]EGTFTSDYSSYLEEQAAKEFIAWLVKGGG GGGGSGGGGSGGGGSK (SEQ ID NO: 14) is bound to cysteine 275 of SEQ ID NO:1 in the AMG 133 molecule.

The anti-GIPR antibodies used in the methods described herein can be monoclonal antibodies, polyclonal antibodies, recombinant antibodies, human antibodies, humanized antibodies, or chimeric antibodies. In certain embodiments, the anti-GIPR antibody is a monoclonal antibody. In such embodiments, the anti-GIPR antibody may be a human monoclonal antibody. In some embodiments, the anti-GIPR antibody is a human antibody and can be of the IgG1-, IgG2-, IgG3-, or IgG4-type. Thus, the anti-GIPR antibody may, in some embodiments, have a human IgG1 or human IgG2 constant domain. In one embodiment, the anti-GIPR antibody is a monoclonal IgG1 antibody. In another embodiment, the anti-GIPR antibody is a monoclonal IgG2 antibody.

Monoclonal antibodies may be produced using any technique known in the art, e.g., by immortalizing spleen cells harvested from the transgenic animal after completion of the immunization schedule. The spleen cells can be immortalized using any technique known in the art. e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

The antagonistic anti-GIPR antibody conjugated to a GLP-1R agonist is generally administered to the patient in a pharmaceutical composition, which can include pharmaceutically-acceptable carriers, excipients, or diluents. “Pharmaceutically-acceptable” refers to molecules, compounds, and compositions that are non-toxic to human recipients at the dosages and concentrations employed and/or do not produce allergic or adverse reactions when administered to humans. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Methods and suitable materials for formulating molecules for therapeutic use are known in the pharmaceutical arts, and are described, for example, in REMINGTON'S PHARMACEUTICAL SCIENCES, 18th Edition, (A. R. Gennno, ed.), 1990, Mack Publishing Company.

In some embodiments, the selection of carriers and excipients for incorporation into the pharmaceutical compositions influences the physical state, stability, rate of in vivo release and rate of in vivo clearance of the anti-GIPR antibodies or binding fragments thereof. In certain embodiments, the primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration.

In certain embodiments of the methods described herein, the antagonistic anti-GIPR antibody conjugated to a GLP-1R agonist is administered to the patient parenterally. Parenteral administration includes intraperitoneal, intramuscular, intravenous, intraarterial, intradermal, subcutaneous, intracerebral, intracerebroventricular, and intrathecal administration. In one particular embodiment, the pharmaceutical composition comprising a therapeutically effective amount of an antagonistic anti-GIPR antibody conjugated to a GLP-1R agonist is administered to the patient subcutaneously. In these and other embodiments in which the pharmaceutical composition is administered by parenteral injection, the pharmaceutical composition can be administered to the patient with a syringe. In some embodiments, the syringe is pre-filled with the pharmaceutical composition. In other embodiments in which the pharmaceutical composition is administered to the patient by parenteral injection, such as subcutaneous injection, the pharmaceutical composition is administered with an injection device, including devices for self-administration. Such devices are commercially available and include, but are not limited to, autoinjectors, dosing pens, microinfusion pumps, and pre-filled syringes. Exemplary devices for administering a pharmaceutical composition comprising a therapeutically effective amount of an antagonistic anti-GIPR antibody conjugated to a GLP-1R agonist according to the methods of the invention include autoinjectors (e.g., SureClick®, EverGentle®, Avanti®, DosePro®, Molly®, and Leva®), pen injection devices (e.g., Madie® pen injector, DCPT™ pen injector, BD Vystra™ disposable pen. BD™ reusable pen), and pre-filled syringes (BD Sterifill™, BD Hvpak™, prefilled syringes from Baxter). In some embodiments, the pharmaceutical composition comprising a therapeutically effective amount of an antagonistic anti-GIPR antibody conjugated to a GLP-1R agonist is administered to the patient with a pre-filled syringe. In other embodiments, the pharmaceutical composition comprising a therapeutically effective amount of an antagonistic anti-GIPR antibody conjugated to a GLP-1R agonist is administered to the patient with an autoinjector. In certain related embodiments, the injection volume is about 1 mL or less.

In one embodiment, the antagonistic anti-GIPR antibody conjugated to a GLP-1R agonist is administered to a patient at a dose of about 280 mg every month to promote weight loss in the patient, wherein the dose is delivered by a single subcutaneous injection. In related embodiments, the single subcutaneous injection is delivered with a pre-filled syringe. In other related embodiments, the single subcutaneous injection is delivered with an autoinjector.

In another embodiment, the antagonistic anti-GIPR antibody conjugated to a GLP-1R agonist is administered to a patient at a dose of about 280 mg every month to promote weight loss in the patient, wherein the dose is delivered by a single subcutaneous injection. In such embodiments, the single injection may be delivered with a pre-filled syringe or autoinjector. In one embodiment, the 280 mg monthly dose of antagonistic anti-GIPR antibody conjugated to a GLP-1R agonist is administered to the patient through two consecutive injections each comprising a 140 mg dose. In such embodiments, the two consecutive injections can be delivered using two pre-filled syringes or two autoinjectors, each of which contains a 140 mg dose.

Illustrative pharmaceutical forms suitable for parenteral injection include sterile aqueous solutions or dispersions and sterile powders for the extemporaneous preparation of sterile injectable solutions or dispersions. Preferably, the pharmaceutical form is sterile and is sufficiently fluid to allow for delivery via a syringe (i.e., the formulation is not excessively viscous so as to prevent passage through a syringe). Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions can be placed into a container having a sterile access port, for example, an intravenous solution bag or vial having a stopper pierceable by a hypodermic injection needle. Parenteral compositions can also be stored in syringes, autoinjector devices, or pen injection devices or cartridges adapted for use with such injection devices.

In certain embodiments, a pharmaceutical composition useful for treating obesity or type II diabetes according to the methods described herein comprises about 21 mg/ml to about 840 mg/ml of antagonistic anti-GIPR antibody conjugated to a GLP-1R agonist thereof, about 8 mM to about 20 mM sodium acetate, about 0.002% to about 0.0150% weight/volume (w/v) polysorbate, and about 7% to about 10% w/v sucrose. In other embodiments, the pharmaceutical composition comprises about 70 mg/ml to about 280 mg/ml of antagonistic anti-GIPR antibody conjugated to a GLP-1R agonist thereof, about 10 mM to about 15 mM sodium acetate, about 0.008% to about 0.012% w/v polysorbate, and about 8% to about 9% w/v sucrose. The pH of these compositions is in the range of about 4.8 to about 5.5 (e.g., pH of about 4.8, about 5.0, about 5.2, or about 5.4).

In one embodiment, a pharmaceutical composition to be administered according to the methods of the invention comprises about 280 mg/ml antagonistic anti-GIPR antibody conjugated to a GLP-1R agonist thereof, about 10 mM sodium acetate, about 0.004% w/v polysorbate 20, and about 9% w/v sucrose at a pH of 5.2±0.2. In another embodiment, the pharmaceutical composition comprises about 280 mg/ml antagonistic anti-GIPR antibody conjugated to a GLP-1R agonist thereof, about 10 mM sodium acetate, about 0.004% w/v polysorbate 80, and about 9% w/v sucrose at a pH of 5.2±0.2. In another embodiment, the pharmaceutical composition comprises about 140 mg/ml antagonistic anti-GIPR antibody conjugated to a GLP-1R agonist thereof, about 10 mM sodium acetate, about 0.004% w/v polysorbate 20, and about 9% w/v sucrose at a pH of 5.2≤0.2. In yet another embodiment, the pharmaceutical composition comprises about 140 mg/ml antagonistic anti-GIPR antibody conjugated to a GLP-1R agonist thereof, about 10 mM sodium acetate, about 0.004% w/v polysorbate 80, and about 9% w/v sucrose at a pH of 5.2±0.2. In another embodiment, the pharmaceutical composition comprises about 420 mg/ml antagonistic anti-GIPR antibody conjugated to a GLP-1R agonist thereof, about 10 mM sodium acetate, about 0.010% w/v polysorbate 20, and about 9% w/v sucrose at a pH of 5.2±0.2. In still another embodiment, the pharmaceutical composition comprises about 420 mg/ml antagonistic anti-GIPR antibody conjugated to a GLP-1R agonist thereof, about 10 mM sodium acetate, about 0.010% w/v polysorbate 80, and about 9% w/v sucrose at a pH of 5.2±0.2.

The present invention also includes kits for treating obesity or type II diabetes in a patient in need thereof. In one embodiment, the kit comprises a pharmaceutical composition of an antagonistic anti-GIPR antibody conjugated to a GLP-1R agonist described herein and packaging material that provides instructions regarding the use of the pharmaceutical compositions. The pharmaceutical composition of the kit may be present in a container, such as a vial or syringe. The pharmaceutical composition may be provided as a solution, suspension, gel, emulsion, solid, crystal, or as a dehydrated or lyophilized powder. In embodiments in which the pharmaceutical composition is provided as a powder, the kit may also comprise diluents (e.g. water, saline, phosphate-buffer saline) necessary to reconstitute the pharmaceutical composition as well as instructions for preparing the composition for administration. In some embodiments, the kits comprise an injection device for self-administration (e.g. pre-filled syringe or autoinjector) pre-filled with the pharmaceutical composition as described herein. Any of the pre-filled syringes and autoinjectors described above can be included in kits.

The following examples, including the experiments conducted and the results achieved, are provided for illustrative purposes only and are not to be construed as limiting the scope of the appended claims.

### EXAMPLES

### Example 1

### Materials:

### Cell Lines

CHOK1 stably expressing hGLP-IR cells or mouse GLP-1R cells were cultured in Ham's F12 media (Thermo Fisher, Waltham, MA) supplemented with 1% penicillin/streptomycin/L-glutamine (PSG, Thermo Fisher), 10% Fetal Bovine Serum (FBS, Thermo Fisher), 250 μg/ml zcocin (Thermo Fisher). CHO AMID cells stably expressing monkey or rat GLP-1R cells were cultured in Dulbecco's Modified Eagle's Medium (DMEM, Thermo Fisher) supplemented with 1% PSG, 10% dialyzed FBS, 1% non-essential amino acids (NEAA, Thermo Fisher), 1 mM sodium pyruvate, 1% sodium hypoxanthine and thymidine supplement (HT supplement, Thermo Fisher), 400 μg/ml hygromycin (Thermo Fisher). HEK 293T hGIPR cells were cultured in DMEM, 1% PSG, 10% FBS, 5 μg/ml puromycin. CHO AMID mouse or rat GIPR cells were cultured in DMEM supplemented with 1% PSG, 10% dialyzed FBS, 1% NEAA, 1 mM sodium pyruvate, 1% HT supplement, 400 μg/ml hygromycin. 293T monkey GIPR cells were cultured in DMEM supplemented with 1% PSG, 10% FBS, 2 μg/ml puromycin. All cells were cultured in humidified incubators maintained at 37° C. and 5% CO2.

### Normal Mice

Pharmacokinetic evaluation in normal mice was performed at Amgen, Inc. (Thousand Oaks, CA). All study procedures were approved by the Amgen, Inc. Institutional Animal Care and Use Committee and conducted in accordance with the Guide for the Care and Use of Laboratory Animals, 8th edition. Animals used on study were male CD-1 mice 8 to 12 weeks of age and weighing approximately 30 g (Charles River Laboratories, CA). Following a 1-week acclimation, mice (n=3) received a single IV bolus dose of AMG 133 at 5 mg/kg via the lateral tail vein. Blood samples were collected by submandibular venipuncture at predetermined time points up to 14 days after the dose. Whole blood was placed into Microvette® 500 μl K3 EDTA plasma separator tubes (20.1341.102. Sarstedt, Newton, NC), gently mixed by 8-10 manual inversions, and centrifuged at 11,500×g at 4° C. for 5 minutes. The resulting plasma was stored at −70° C. (10° C.) until analysis.

### DIO Mice

### Normal Cynomolgus Monkeys

The PK study in normal monkeys was performed at MPI Research (Mattawan, MI). Animal care was in accordance with the Guide for the Care and Use of Laboratory Animals, 8th Edition, and the study was conducted per protocols approved by the Institutional Animal Care and Use Committee at MPI Research. Animals used on study were females weighing from 2 to 3 kg (young adults) from the MPI Research stock colony of naïve cynomolgus monkeys (Macaca fascicularis). Prior to assignment to study, monkeys were quarantined and acclimated per MPI Research procedures. Monkeys were housed individually in stainless steel cages and were provided environmental enrichment during the study. Lighting was provided via automatic timer for approximately 12 hours per day. Food was offered twice daily (Lab Diet® Certified Primate Diet #5048, PMI Nutrition International) and water was available ad libitum. Temperature and humidity were maintained in the range of 64° F.-79° F. and 30%-70%, respectively. Following an 8-hour fasting period prior to dosing, monkeys (n=3) received a single SC bolus dose of AMG 133 at 3 mg/kg in the scapular region on the back of each animal. Blood samples (˜1 mL) were collected from the femoral vein/artery at predetermined time points up to 35 days after the dose. Blood samples were processed to K2 EDTA plasma and stored at −70° C. (±10° C.) until analysis.

### Obese Cynomolgus Monkeys

### Methods Details:

Peptide Synthesis: Peptides with linkers were prepared by standard fluorenylmethoxycarbonyl (Fmoc)-based solid peptide synthesis using Rink Amide MBHA resin (Peptide International) on an Intavis MultiPep Rsi synthesizer. 20% 4-methyl piperidine in N,N-dimethylforamide (DMF) was used for Fmoc removal and 1,3-diisopropylcarbodiimide (DIC)/ Ethyl cyanohydroxyiminoacetate (Oxyma) were used for amino acid coupling. Boc-Tyr(OtBu)-OH was utilized for the final coupling in the sequence. Each residue was coupled with an excess of coupling solution (5.0 eq), and each coupling reaction was performed twice at each position. The lysine residue was protected with 4,4-dimethyl-2,6-dioxocyclohex-1-ylidene)-3-methylbutyl (ivDde) and the ivDde group was selectively removed with 2% hydrazine in DMF. Bromo acetyl group was introduced with DIC (10 eq)/bromoacetic acid (20 eq) in a mixture of methylene chloride and DMF.

In Vitro cAMP Assay

For GLP-1R agonist activity, CHO cells stably expressing human, mouse, rat and monkey GLP-1R, were used to measure peptide or bispecific molecule-induced cAMP production in a homogeneous time-resolved fluorescence (HTRF) assay (Cisbio, Bedford, MA, cat #62AM4PEJ). Serial diluted peptides or bispecific molecules were incubated with 40,000 cells in assay buffer (0.1% bovine serum albumin, 500 μM 3-Isobutyl-1-methylxanthine in F12 media) for 15 minutes at 37° C. Cells were then lysed with lysis buffer containing cAMP-d2 and cAMP cryptate (Cisbio, Bedford, MA) and incubated for 1 hour at RT before measurement in the Envision plate reader (PerkinElmer, Waltham, MA). The cAMP levels are expressed as a fluorescence ratio of 665/620 nm.

For GIPR antagonist activity, HEK 293T cells stably expressing human or monkey GIPR and CHO AMID stably expressing mouse GIPR were used to measure peptide or bispecific molecule-induced cAMP production in a HTRF assay. Serial diluted molecules were incubated with 30,000 cells in assay buffer (0.1% BSA, 500 μM IBMX in F12 media) for 30 minutes at 37° C. before treatment with GIP at final concentration of 0.05 nM. Cells were incubated for 30 minutes at 37° C. and then lysed in lysis buffer containing cAMP-d2 and cAMP cryptate (Cisbio) for 1 hour at RT. The fluorescence was measured in an Envision plate reader (PerkinElmer), and the cAMP levels are expressed as a ratio of 665/620 nm.

The graphs were generated by plotting the concentration of GLP-1, GIP, AMG 133, or AMG 133 surrogate on the x-axis, against the average of two replicate cAMP values with the standard error mean on the y-axis. The dose response curves were then analyzed using log of agonist or antagonist versus response, variable slope (4 parameters) GraphPad Prism fit, to calculate both EC50 and IC50 values.

### Bioanalytical Methods and Pharmacokinetics

Concentrations of AMG 133 in mouse and cynomolgus monkey plasma were determined by ELISA methods developed to monitor intact AMG 133 (anti-GIPR-Ab with attached GLP-1 analog peptides) and total AMG 133 (anti-GIPR-Ab with or without GLP-1 analog peptides). The analytical range for both assays was from 30 to 2000 ng/mL. Pharmacokinetic parameters were estimated from individual plasma concentration-nominal time data using standard noncompartmental analysis in Phoenix® WinNonlin® (v6.4; Certara, Princeton, NJ).

### Intact AMG 133 ELISA

Microtiter plates were passively coated with a mouse mAb directed against human IgG Fe (clone no. 1.35, Amgen Inc.) in phosphate-buffered saline overnight at 4° C. Coated plates were blocked with blocking buffer overnight at 4° C. Calibration standards were prepared in a range from 30 to 2,000 ng/mL in mouse or monkey plasma (BioIVT, Westbury, NY). After dilution in blocking buffer, standards, controls, and unknown samples were added and incubated for ˜2 hours at RT. After washing, a biotin-conjugated mouse mAb directed against free N-terminus of GLP-1 (clone no. 4, Thermo Fisher) was added and incubated for ˜1 hour at RT. Following an additional wash step, a streptavidin-horseradish peroxidase (SAV-HRP) conjugate (R&D Systems, Inc., Minneapolis, MN) was added and incubated for ˜30 minutes at RT. After a final wash step, a tetramethylbenzidine peroxide substrate solution (SeraCare, Milford, MA) was added and incubated for ˜10 minutes at RT. The chromogenic reaction was stopped by addition of H2SO4, and absorbance values were determined at 450 nm with reference to 650 nm using a SpectraMax microtiter plate reader (Molecular Devices, San Jose, CA). Sample concentrations were interpolated from a standard curve fit to a four-parameter logistic model using Watson LIMS (v7.4: Thermo Fisher).

### Total AMG 133 ELISA

The total assay followed a similar procedure as the intact assay with the exception of the detection reagent. Detection of AMG 133 in the total assay was achieved with an HRP-conjugated mouse monoclonal antibody against human IgG Fe (Clone No 21.1, Amgen, Inc., CA).

### Example 2

### Phase 1 Clinical Study Details:

### Clinical Study Design and Randomization

This was a first-in-human, double-blind, randomized, placebo-controlled study comprised of two parts: single ascending dose (SAD) and multiple ascending dose (MAD) in participants with obesity. Three clinical sites in the United States are conducting this ongoing trial starting from August 2020. The study (ClinicalTrials.gov, NCT04478708) was conducted in full accordance with the Declaration of Helsinki and the International Conference on Harmonisation Good Clinical Practices Guideline. The study protocol, all amendments, and the informed consent form were reviewed and approved by the Institutional Review Board at each site. Written informed consent was obtained by eligible participants prior to any study-related procedures.

Eligible participants were women of non-reproductive potential and men aged 18 to 65 years with a body mass index (BMI) between 30.0 kg/m2 and 40.0 kg/m2 and HbA1c≤6.5% and/or a fasting glucose of ≥125 mg/dl. Participants had normal vital signs, 12-lead electrocardiogram results and clinical laboratory tests at the time of randomization.

Participants were randomly assigned to receive AMG133 or placebo in a ratio of 3:1. The SAD evaluated AMG 133 doses ranging from 21-840 mg in six cohorts. The MAD evaluated 3 doses given every 4 weeks on Days 1, 29 and 57. The sentinel pair was observed for at least 48 hours before the remaining participants were dosed, provided there were no safety or tolerability concerns as assessed by the investigator. Enrollment into the SAD and MAD cohorts was sequential. Subsequent cohorts were dosed after the dosing regimen in the preceding cohort had been recommended by the Dose Level Review Team (DLRT) to be safe and well tolerated based on the safety and laboratory data through at least day 15. Participants received AMG 133 in vials containing 70 mg/ml of AMG 133. Placebo was provided as normal saline, the volume matching the investigational drug at each dose level. AMG 133 or matching placebo was administered in the morning (fasting) by subcutaneous injection in the abdomen of the participant.

### Clinical Outcomes

The primary endpoints were the participant incidence of treatment-emergent adverse events, changes in laboratory safety tests, vital signs, and 12-lead electrocardiograms. The secondary endpoints were AMG 133 pharmacokinetic parameters including, but not limited to, maximum observed drug concentration during a dosing interval (Cₘₐₓ), the time of maximum observed concentration (tmax), and area under the concentration-time curve (AUC). The exploratory endpoints were pharmacodynamic parameters including, but not limited to, concentrations of fasting glucose, insulin, c-peptide, glucagon,, hemoglobin A1c, changes in body weight, waist circumference, and BMI.

### Statistical Analysis for Phase 1 Study

The sample size for this trial was not based on any statistical inferences. As the trial was an exploratory first-in-human study with a small sample size, all pharmacokinetic, pharmacodynamic and safety parameters were analyzed descriptively. Participants who withdrew from the study prior to day 15 in the SAD or day 36 in the MAD were replaced so that the target numbers of participants for safety review and data collection would be achieved; replacement participants were assigned to receive the same treatment as the withdrawn participant. The full analysis set consisted of all randomized participants who receive at least 1 dose of AMG 133. The safety analysis set was the same as the full analysis set. The pharmacokinetics analysis set consisted of all participants who received at least 1 dose of AMG 133 for whom at least 1 pharmacokinetics parameter or endpoint could be adequately estimated. No missing data was imputed.

### Inclusion Criteria

Participants are eligible to be included in the study only if all of the following criteria apply:
Participant had provided informed consent before initiation of any study-specific activities/procedures.
Age ≥18 years to ≤65 years at the time of signing the informed consent
Except for obesity, otherwise healthy based on a medical evaluation including medical history, physical examination, laboratory tests, and ECGs
BMI between ≥30.0 kg/m2 and ≤40.0 kg/m2
Had a stable body weight (<5 kg self-reported change during the previous 8 weeks) before screening
Willing to maintain current general diet and physical activity regimen, except for the physical activity in the 72 hours before each blood sample collection for the clinical laboratory analysis, which should not be strenuous.
Females must be of nonreproductive potential
Postmenopausal defined as age of ≥55 years with no menses for at least 12 months or age <55 years with no menses for at least 12 months and with a follicle-stimulating hormone level >40 IU/L
History of hysterectomy
History of bilateral oophorectomy
Exclusion criteria
Participants are excluded from the study if any of the following criteria apply:
History or clinical evidence of diabetes mellitus, including HbA1c >6.5% and/or a fasting glucose ≥125 mg/dl (6.9 mmol/L) at screening
Triglycerides ≥5.65 mmol/L (ie, 500 mg/dL) at screening
Screening calcitonin ≥50 ng/L
Hepatic liver enzymes aspartate aminotransferase (AST), alanine aminotransferase (ALT), alkaline phosphatase, or total bilirubin levels >1.5 times the upper limit of normal (ULN) at screening
History or clinical evidence of bleeding diathesis or any coagulation disorder, including prothrombin time (PT), activated partial thromboplastin time (PTT), international normalized ratio (INR), or platelet count outside of the laboratory's normal reference range at screening.
History of gastrointestinal abnormality that could affect gastrointestinal motility (including small bowel or colonic resection, inflammatory bowel disease, irritable bowel disease, and colon or gastrointestinal tract cancer)
A family or personal history of medullary thyroid carcinoma or multiple endocrine neoplasia type 2 or a personal history of nonfamilial medullary thyroid carcinoma
History of confirmed chronic pancreatitis or idiopathic acute pancreatitis
History of gall bladder disease (ie, cholelithiasis or cholecystitis) not treated with cholecystectomy
Untreated or uncontrolled hypothyroidism/hyperthyroidism defined as thyroid-stimulating hormone >6 mIU/L or <0.4 mIU/L
A corrected QT interval (QTc) at screening of >450 msec in males or >470 msec in females or history of long QT syndrome
History of renal impairment or renal disease and/or estimated glomerular filtration rate <60 mL/min/1.73 m2
Obesity induced by other endocrinologic disorders (eg, Cushing's Syndrome)
Previous surgical treatment for obesity (excluding liposuction if performed >1 year before study entry) and/or subjects with recent (within 6 months) or planned endoscopic treatment for obesity
History of major depressive disorder, severe psychiatric disorders, or any suicidal behavior
Positive results for human immunodeficiency virus antibodies, hepatitis B surface antigen, hepatitis B core antibody, or hepatitis C virus RNA
Systolic blood pressure ≥150 mm Hg or diastolic blood pressure ≥90 mm Hg at screening
Surgery scheduled for the study duration period, except for minor surgical procedures
History of malignancy of any type, other than in situ cervical cancer or surgically excised nonmelanomatous skin cancers occurring more than 5 years before randomization

### Results

### AMG 133 PK and PD in Human

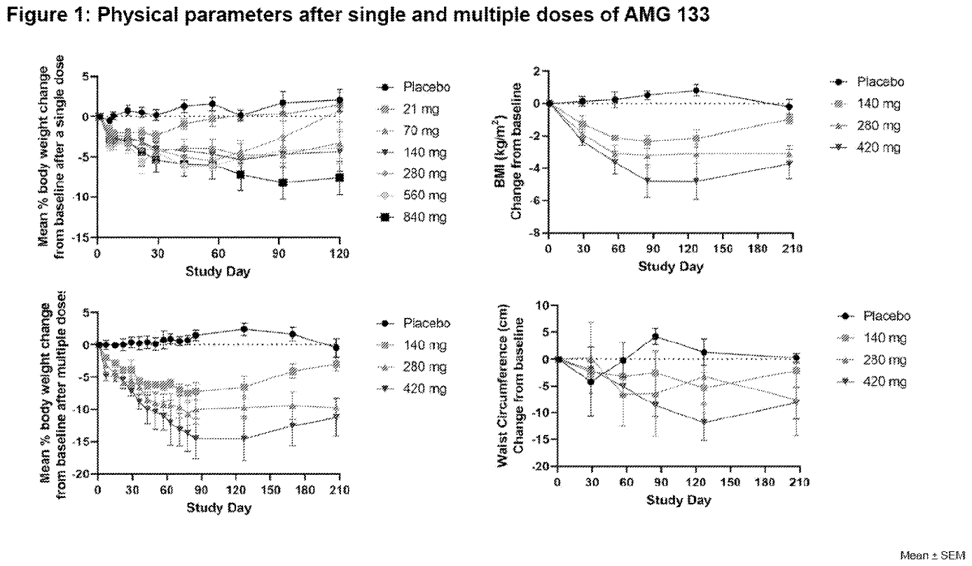

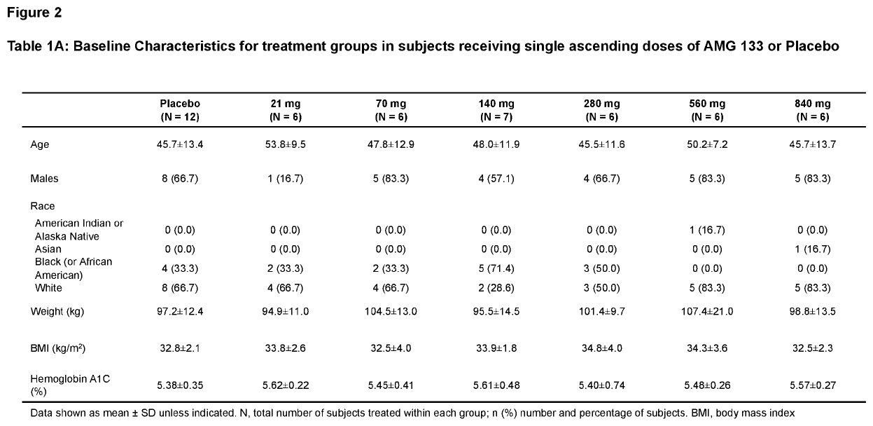

A phase 1, randomized, double-blind, placebo-controlled study was conducted to evaluate the safety, tolerability, PK, and PD of single ascending doses (SADs) and multiple ascending doses (MADs) of AMG 133 (NCT04478708). A total of 75 adults with obesity enrolled in the study: 49 participants entered in the SAD part (6 cohorts); 26 participants entered in the MAD part (3 cohorts) and received AMG 133 every 4 weeks for 12 weeks (85 days treatment period) with post-dose follow-up until day 207. One participant in the 140 mg SAD cohort and 3 participants in the 420 mg MAD cohort withdrew consent within the first 29 days of the study (FIG. 1). The baseline demographic characteristics are summarized in Table 1. The mean age ranged from 45.7 to 50.2 years in the SAD cohorts and 40.3 to 51.6 years in the MAD cohorts. The mean BMI ranged from 32.5 to 34.8 kg/m2 in the SAD cohorts and 32.5 to 34.2 kg/m2 in the MAD cohorts. The participants did not have a history of diabetes mellitus and the mean HbA1c ranged from 5.38 to 5.61% in the SAD cohorts and 5.50 to 5.58% in the MAD cohorts.

The pharmacokinetics of AMG133 appeared to be dose-proportional over the range of doses evaluated from 21 mg to 840 mg. The mean half-life is approximately 14 to 25 days, thus supporting dosing of AMG133 every 4 weeks. Treatment with AMG 133 at all dose levels (SADs and MADs) resulted in decreases in mean body weight from baseline (FIG. 1). Reductions in body weight were greatest at the highest dose levels of SAD 840 mg and MAD 420 mg. At the lowest single dose of 21 mg, the mean percent change of body weight from baseline ranged from −1.59% at post-dose day 6 to a maximal decrease of 2.39% at day 29. The highest single dose of 840 mg resulted in a −2.85% change at day 6 and a maximal decrease of 8.24% at day 92, suggesting a prolonged weight-reducing effect after a single dose of AMG 133. The decreases in body weight were maintained until day 150 following all SADs except the lowest dose 21 mg. The lowest MAD dose of 140 mg reduced 1.97% of the body weight from baseline at post-dose day 7 and reached a maximal reduction of 7.44% after 3 doses at day 78. The highest MAD dose of 420 mg resulted in a decrease in body weight by −4.86% by day 7 and −14.5% by day 85. The reduction in body weight after multiple doses of AMG 133 was maintained until end of study day 207 in the MAD cohorts. The change from baseline in BMI followed a similar pattern as the change in body weight (FIG. 1). A dose-dependent decrease in waist circumference was observed with MAD 140 mg and 420 mg whereas fluctuations in waist circumference changes were displayed with MAD 280 mg.

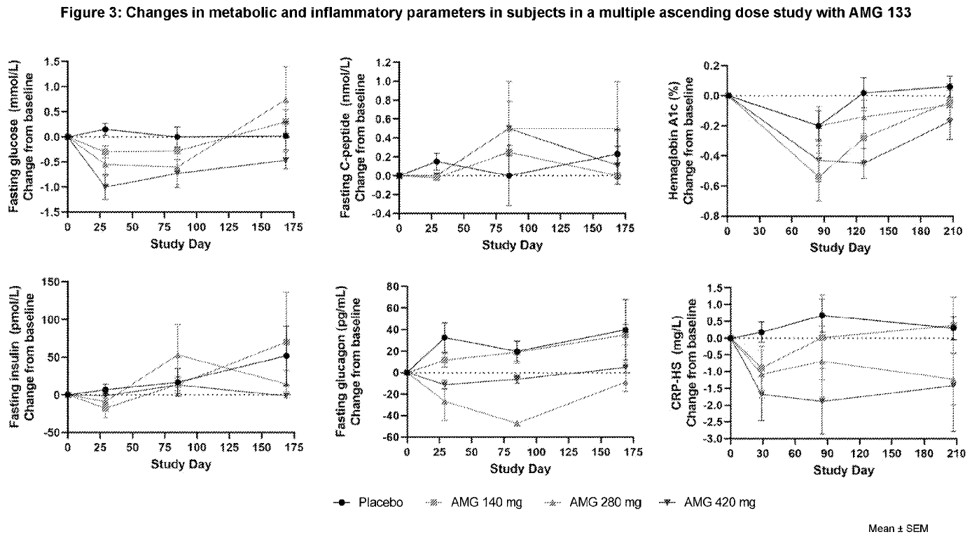

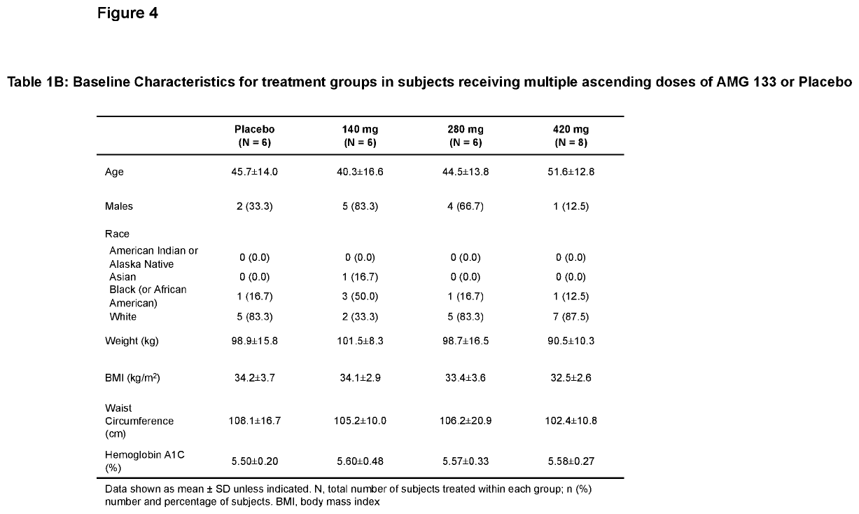

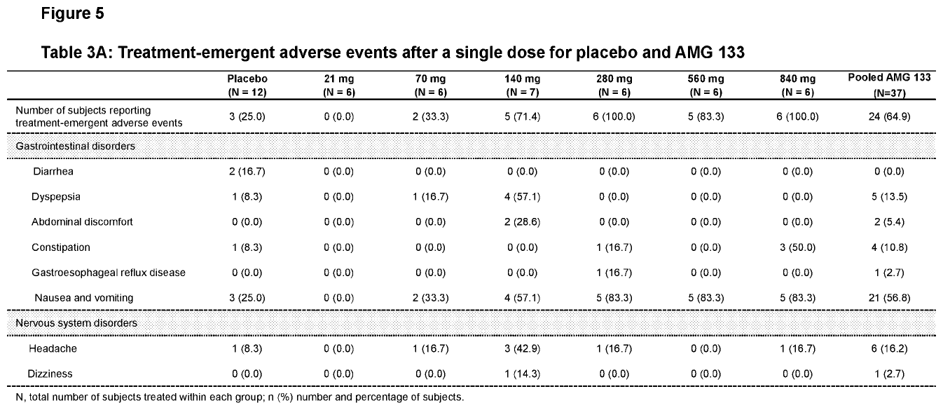

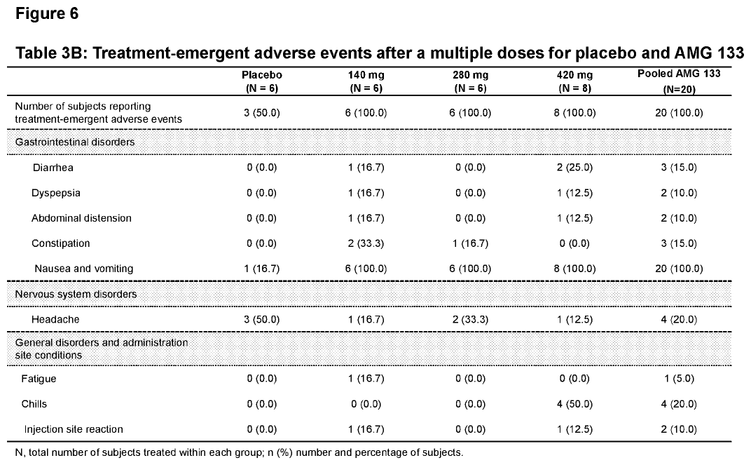

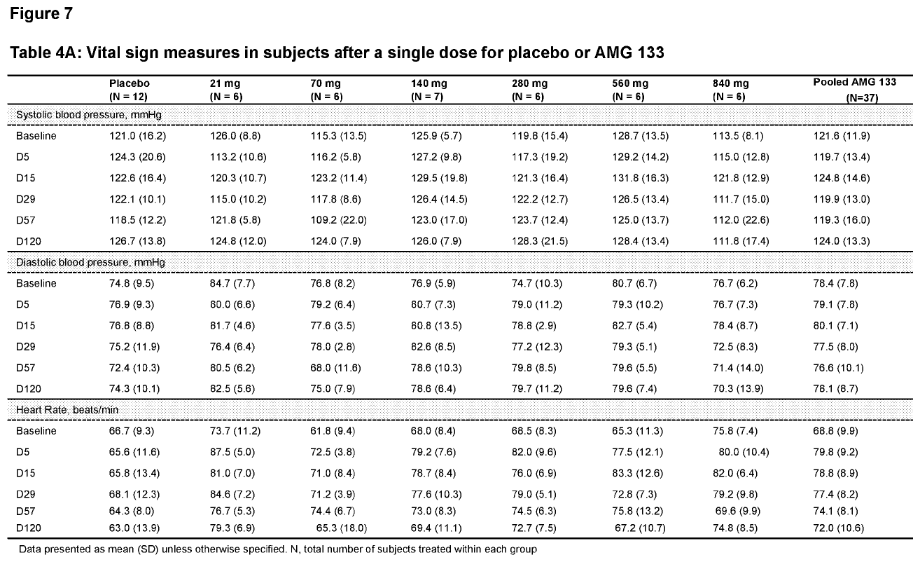

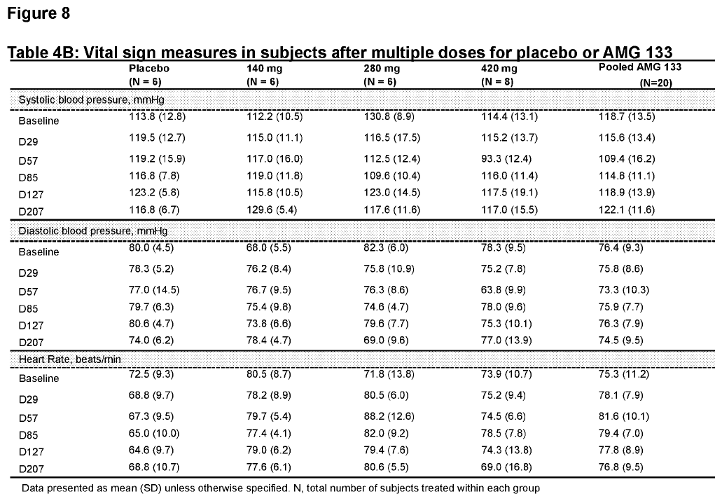

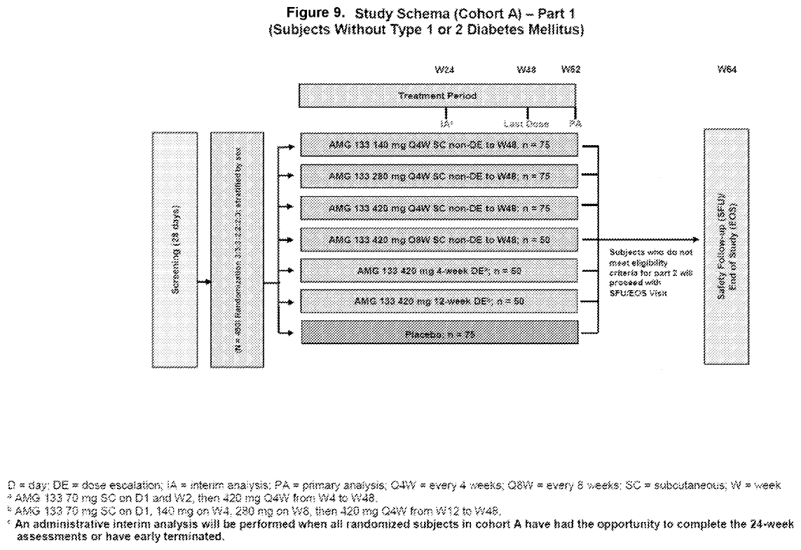

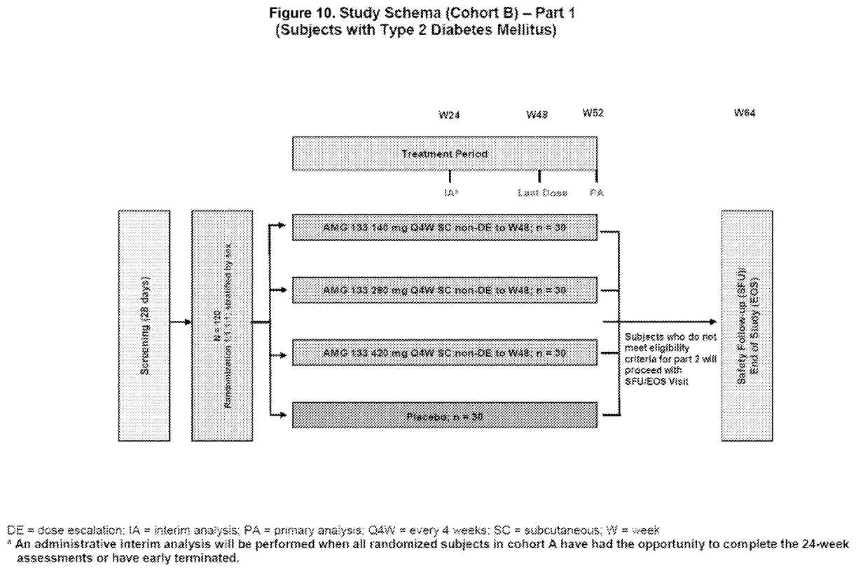

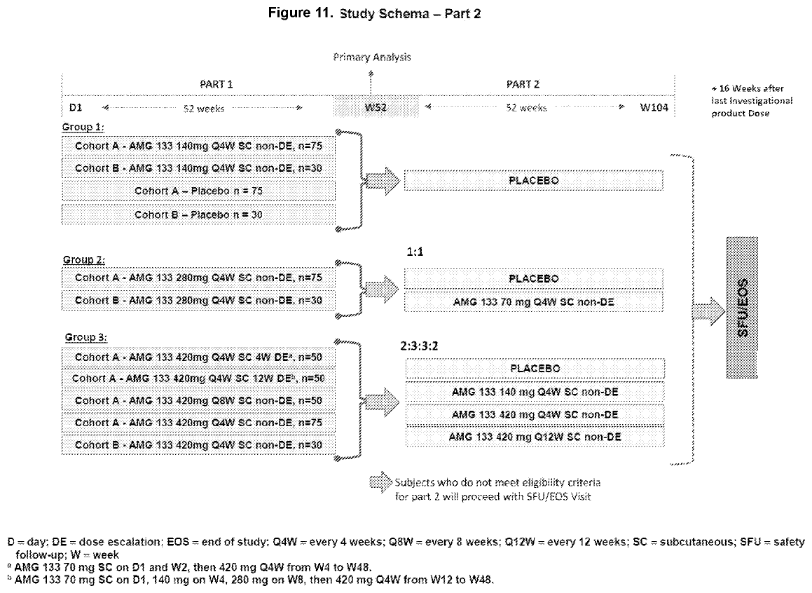

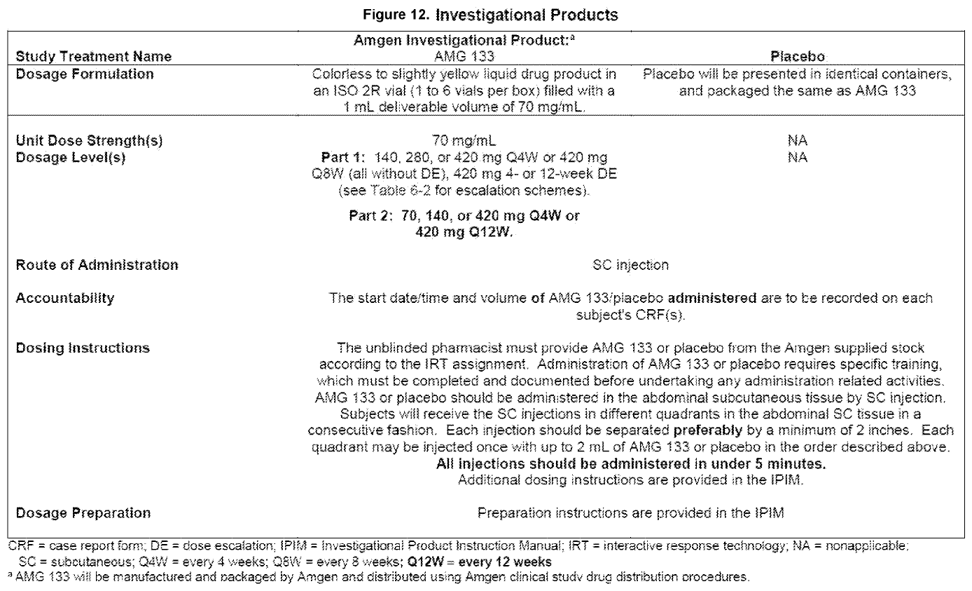

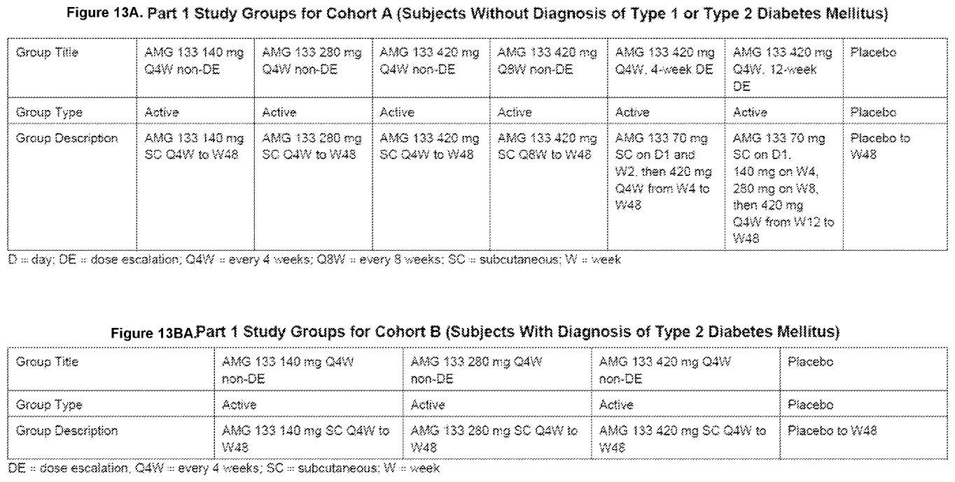

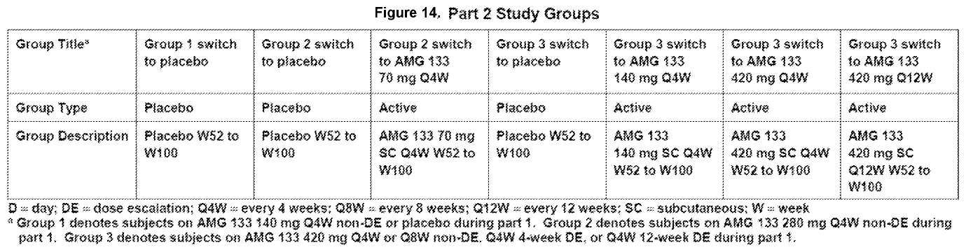

Changes of the pharmacodynamic makers in the MAD cohorts are shown in FIG. 3. Following treatment with 140 and 420 mg AMG 133, the fasting glucose decreased in a dose-responsive manner at day 29 and 85. The lower glucose levels were maintained throughout the safety follow up period (day 127) at the 420 mg dose but the glucose levels returned to above baseline at the 140 and 280 mg doses. The greatest reduction in glucagon was observed at day 85 which normalized by day 127 at the 280 mg dose. HbA1c levels which were in the normal (non-diabetic) range at baseline, decreased in all three dose groups but trended towards baseline at day 127. There was a dose-responsive decrease in hs-CRP from baseline among MAD cohorts. The reductions in hs-CRP levels were sustained until day 127 at 280 and 420 mg doses but normalized to baseline at 140 mg dose.

Participants tolerated AMG 133 well. No serious TEAEs, TEAEs leading to permanent discontinuation, or severe TEAEs were reported during the study (Table 3A and 3B). In the SAD cohorts, all GI-related TEAEs were mild except one participant in the 140 mg cohort had moderate GI symptoms. Dyspepsia was more frequently reported with lower doses (70 mg and 140 mg) treatment whereas nausea and vomiting were reported in participants treated with ≥70 mg AMG 133. In the MAD cohorts, all participants who received AMG 133 experienced mild nausea and vomiting. No meaningful changes in blood pressure were observed after either a single dose or multiple doses of AMG 133 (Table 4A and 4B). The heart rate increased with a single dose of 280, 560 or 840 mg AMG 133 but not in a dose-dependent manner. In the MAD cohorts, the 280 mg cohort had higher heart rates than the 140 mg or 420 mg cohort suggesting the heart rate change was not dose dependent.

### Study DesignStudy DesignStudy Design

### Overall DesignOverall DesignOverall Design

Part A (cohorts 1 to 6 and cohort 11) is a phase 1, randomized, double-blind, placebo-controlled, SAD study in adult subjects with obesity. AMG 133 will be administered SC for cohorts 1 to 5 and cohort 11, and IV for cohort 6. Part A consists of a total of 7 cohorts.

Subjects will be confined at the Clinical Research Unit from check-in (morning of day −2) through the morning of day 8 for cohorts 1 to 5 and cohort 11 and, day 6 for cohort 6.

Approximately 56 subjects will enroll into 1 of 7 cohorts. In each cohort, 8 subjects will be randomized to receive AMG 133 or placebo SC (cohorts 1 to 5 and cohort 11) or IV (cohort 6) in a 3:1 ratio as described in Table 1. For cohort 1, the first 2 subjects (sentinel pair) will be randomized such that 1 subject will receive AMG 133 and 1 subject will receive placebo. The sentinel pair will be observed for at least 48 hours before the remaining subjects in the cohort are dosed, provided there are no safety or tolerability concerns as assessed by the investigator. Enrollment into the SAD cohorts will be sequential. Subsequent cohorts will be dosed after the dosing regimen in the preceding cohort has been recommended by the Dose Level Review Team (DLRT) to be safe and well tolerated based on the safety and laboratory data through at least day 15 for at least 7 out of 8 subjects dosed. Subjects in cohorts 1 to 5 and cohort 11 will also participate in gastric emptying tests at day −1 and day 7.

Part B (cohorts 7 to 10) is a randomized, placebo-controlled, double-blind, MAD study in adult subjects with obesity. In each cohort, subjects will be randomized to receive AMG 133 or placebo SC in a 3:1 ratio as described in Table 1. Approximately 24 subjects will enroll into cohorts 7 to 9. Cohorts 7 to 9 will include assessments of food intake with the use of the Remote Food Photography Method (RFPM). Enrollment into Part B cohort 7 will occur with a starting MAD dose that is at least 2 SAD dose levels below what was recommended by the DLRT to be safe and reasonably tolerated in Part A. Enrollment into the MAD cohorts 8 and 9 will be sequential.

Subjects in cohorts 8 and 9 will be dosed after the dose regimen in the preceding MAD cohort has been recommended by the DLRT to be safe and reasonably tolerated based on safety and laboratory data through at least study day 36 for 6 out of 8 subjects dosed. The DLRT for SAD cohort 11 will also make dosing recommendations for cohort 9. In brief, if the DLRT agrees that the dose for cohort 11 did not result in any safety and tolerability concerns, then the dose recommended for cohort 9 will be ≤560 mg SC Q4W (every 4 weeks). However, if there are safety and tolerability concerns, then the DLRM for cohort 11 may recommended a dose 420 mg SC Q4W for cohort 9. All subjects in each Part B (MAD) cohort may be dosed on the same day.

Cohort 10 will enroll up to 20 subjects and will include the use of digital health tools for an exploratory assessment of sleep, activity, and weight. The purpose of cohort 10 is to assess whether digital tools affect subjects' behavior resulting in additional effects on weight beyond the IP. Therefore, the dose recommended for cohort 10 should be the same as a dose studied in a previous cohort.

The dose recommended for cohort 10 will depend on the final dose recommended for cohort 9 and the safety and tolerability profiles for MAD cohorts 7 and 8. If the DLRT for cohort 7 (≤140 mg SC Q4W) finds an acceptable safety and tolerability profile, then the DLTR may recommend an ascending dose for cohort 8 (<280 mg SC Q4W). However, if the DLRT for cohort 7 finds safety and tolerability concerns, then the DLRT may recommend a dose for cohort 10 (≤140 mg SC Q4W). In this case, cohort 10 enroll sequential to cohort 8. If the DLRT for cohort 8 finds an acceptable safety and tolerability profile, then they may recommend advancing to cohort 9. If the dose recommended by the DLRT for cohort 9 is ≤420 mg SC Q4W, then the dose for cohort 10 will be recommended at the DLRM for cohort 8 and cohort 10 will enroll in parallel to cohort 9. If the dose selected for cohort 9 is ≤560 mg SC Q4W, then the DLRT for cohort 8 may recommended the dose for cohort 10 and cohort 10 may enroll in parallel to cohort 9.

MAD cohorts 7 to 9: Study drug will be administered every 4 weeks for a total of 3 SC doses. The dose levels will be defined after evaluation of the available PK and PD data from preceding cohorts in the SAD phase (Part A). Three different dose levels will be evaluated with the lowest dose administered to cohort 7, and 2 higher ascending doses administered to cohorts 8 and 9. The dose level for cohorts 7 to 9 will not exceed the highest dose evaluated in cohorts 1 to 6 and cohort 11 (Part A). Subjects enrolled in cohorts 7 to 9 will also be asked to record their food intake by using the RFPM.

MAD cohort 10: Up to twenty subjects will be randomized in a 3:1 ratio to receive AMG 133 or placebo SC. All subjects will be asked to use digital health tools such as digital scales and activity/sleep trackers (to be provided by Amgen). The dose level will be defined after evaluation of the available PK and PD data from preceding MAD cohorts. The dose level will not exceed the highest dose evaluated in cohorts 7 to 9 (Part B).

Part C (cohorts 12 to 13) is an open-label modified dose-escalation MAD study in subjects with obesity. Approximately 12 subjects (up to 6 subjects per cohort) will enroll in Part C and receive AMG 133 (see Table 1). Doses selected for these cohorts are known to have an acceptable safety and tolerability profile based on data reviewed by the DLRT from previous cohorts in Part A and Part B and will not exceed the dose levels previously studied in the MAD cohorts 7 to 9. Cohort 12 will receive 70 mg SC on days 1 and 8, followed by 420 mg SC on days 15 and 43. Cohort 13 will receive 70 mg on days 1, 8, 15, and 22 followed by 420 mg SC on days 29 and 57. Cohorts 12 and 13 will enroll in parallel. The DLRT will review the safety and laboratory data through at least day 36.

TABLE 1
Planned Dose Levels by Cohort

| | | No. | AMG 133/Placebo | | N |
|---|---|---|---|---|---|
| | Cohort | Subjects | Dose/Frequency | Route | (active:placebo) |
| Part | 1 | 8 | 21 mg day 1 | SC | 6:2 |
| A | 2 | 8 | Not exceeding 70 mg day 1^{a} | SC | 6:2 |
| | 3 | 8 | Not exceeding 140 mg day 1^{a} | SC | 6:2 |
| | 4 | 8 | Not exceeding 280 mg day 1^{a} | SC | 6:2 |
| | 5 | 8 | Not exceeding 560 mg day 1^{a} | SC | 6:2 |
| | 11 | 8 | Not exceeding 840 mg day 1^{a} | SC | 6:2 |
| | 6 | 8 | Not exceeding 70 mg day 1^{a} | IV | 6:2 |
| Part | 7 | 8 | Not exceeding 140 mg^{b} Q4W × 3 | SC | 6:2 |
| B | 8 | 8 | Not exceeding 280 mg^{b} Q4W × 3 | SC | 6:2 |
| | 9 | 8 | Not exceeding 560 mg^{b} Q4W × 3 | SC | 6:2 |
| | 10 | ≤20 | Not exceeding 560 mg^{c} Q4W × 3 + | SC | ≤15:5 |
| | | | Digital health devices | | |
| Part | 12 | ≤6 | 70 mg on days 1 and 8 followed by | SC | N/A |
| C | | | 420 mg on days 15 and 43 | | |
| | 13 | ≤6 | 70 mg on days 1, 8, 15, and 22 | SC | N/A |
| | | | followed by 420 mg on days 29 and 57 | | |

N/A = not applicable; Q4W = every four weeks; SC = subcutaneous; IV = intravenous.
^{a}Actual dose levels will be based on available data from previous cohorts.
^{b}Dose will not exceed the highest dose evaluated in cohorts 1 to 5 and cohort 11 (Part A)
^{c}Dose will not exceed the highest dose evaluated in cohorts 7 to 9 (Part B)

The overall study design is described by a study schema in Section Error! Reference source not found.. The endpoints are defined in Section Error! Reference source not found..

### Number of Subjects

A total of approximately 112 subjects will be enrolled in the study. Approximately 56 subjects will be enrolled in Part A of the study, with 8 subjects in each of the 7 cohorts. Approximately 44 subjects will be enrolled in Part B of the study, with 8 subjects in cohorts 7 to 9 and up to 20 subjects in cohort 10. Up to 12 subjects will be enrolled in Part C cohorts 12 to 13. Additional subjects may be enrolled if a DLRT recommendation is made to expand, repeat, or add cohorts to the study or if replacement subjects are needed.

Subjects in this clinical investigation shall be referred to as “subjects.”

### Replacement of Subjects

Subjects who withdraw from the study or who discontinue study drug administration prematurely may be replaced at the discretion of Amgen in consultation with the investigator. The replacement subject will be assigned to receive the identical treatment as the replaced subject.

### Number of Sites

Approximately 1 to 3 investigative sites in United States will be included in the study. Sites that do not enroll subjects within 1 month of site initiation may be closed.

### Justification for Investigational Product Dose

The planned cohort in this amendment of the study consists of a multiple-dose cohort following a titration schema.

Previously, subjects in Part A received single doses of AMG 133-21, 70. 140, 280. 560, and 840 mg administered SC. This part also consisted of an IV cohort (cohort 6) at a dose of 70 mg administered IV; all SAD have been completed. The starting dose and exposure multiples for the doses evaluated in SAD were calculated based on the NOAELs determined in the 13-week Good Laboratory Practices repeat-dose mouse and cynomolgus monkey nonhuman primate toxicology studies (every 2 weeks dosing, Section Error! Reference source not found.) and the observed reduction in body weight in the chronic efficacy study in obese cynomolgus monkeys.

Part B consisted of evaluation of AMG 133 following repeat doses −140, 280, and 420 mg administered Q4W for a total of 3 doses per cohort. Preliminary review of the PK data in the FIH SAD and MAD part of the study demonstrated that AMG 133 pharmacokinetics follow a linear, dose proportional kinetics based on Cₘₐₓ (area under the curve [AUC] data pending), with a half-life of approximately 15 to 20 days.

For SAD cohorts 1 to 6 and 11, no serious adverse events or deaths have been reported, and most adverse events reported were mild associated with nausea, vomiting, and dyspepsia. One subject in cohort 3 (140 mg dose) had a moderate adverse event associated with gastroenteritis related to IP. Approximately 90% of subjects enrolled in MAD cohorts 7 to 10 reported mild GI-related adverse events (mainly nausea, vomiting, and diarrhea) after the first dose of AMG 133 and less than 5% experienced GI-related adverse events after the second dose. There were no serious adverse events or deaths reported after any of the doses. Approximately 26% of subjects in the MAD trial discontinued the study prior to receiving the second dose which caused concerns regarding tolerability of the first dose; 50% of subjects in cohort 9 withdrew consent after receiving the 420 mg dose on day 1 due to GI-related adverse events. The doses were selected to allow for whole integer injection volumes to circumvent any potential dosing errors.

Part C consists of two cohorts (cohorts 12 and 13), both open-label to evaluate dose-level escalation from 70 mg to 420 mg. The objective of Part C is to evaluate first dose effects of AMG 133 on GI-related adverse events using a titration methodology. Cohort 12 will start at a lower dose (70 mg) of AMG 133 given weekly in 2 doses in order to build up exposure levels prior to receiving repeat doses of 420 mg on days 15 and 43.

Cohort 13 will start at a lower dose (70 mg) of AMG 133 given weekly for 4 doses in order to build up exposure levels prior to receiving repeat doses of 420 mg on Days 29 and 57.

The rationale for dose selection for Part C is based on the available PK data from this ongoing FIH study cohorts 1 to 11 (Part A and Part B) and the NOAEL exposures established based on 13-week GLP repeat-dose studies in mouse and cynomolgus monkeys.

Similarly, for cohort 12, the predicted human exposure multiples for intact AMG 133 following the second dose of 420 mg on Day 43 are approximately 40.5-fold and 45-fold for Cₘₐₓ and AUCₛₛ, respectively, based on NOAEL established in cynomolgus monkeys, and 43-fold and 42.4-fold for Cₘₐₓ and AUCₛₛ, respectively, based on NOAEL established in mice. The predicted human exposure multiples for total AMG 133 following the second dose of 420 mg on Day 43 are approximately 45-fold and 49-fold for Cₘₐₓ and AUCₛₛ, respectively, based on NOAEL established in cynomolgus monkeys, and 54-fold and 42-fold for Cₘₐₓ and AUCₛₛ, respectively, based on NOAEL established in mice (Table 2 and Table 3).

For cohort 13, the predicted human exposure multiples for intact AMG 133 following the second dose of 420 mg on Day 57 are approximately 39-fold and 43.2-fold for Cₘₐₓ and AUCₛₛ, respectively, based on NOAEL established in cynomolgus monkeys, and 41.3-fold and 40.6-fold for Cₘₐₓ and AUCₛₛ, respectively, based on NOAEL established in mice. The predicted human exposure multiples for total AMG 133 following the second dose of 420 mg on Day 57 are approximately 42.2-fold and 45.5-fold for Cₘₐₓ and AUCₛₛ, respectively, based on NOAEL established in cynomolgus monkeys, and 50.9-fold and 47.5-fold for Cₘₐₓ and AUCₛₛ, respectively, based on NOAEL established in mice (Table 2 and Table 3).

TABLE 2
Estimated Exposure Margins of Intact AMG 133 Following Repeat Doses (Q4W)

| | | Predicted | | Predicted Exposure | | | |
|---|---|---|---|---|---|---|---|
| | | Human Exposure | | Multiples^{a} | | | |
| Clinical | | AUC₀₋₂₈ | Cₘₐₓ | Mouse^{b} | | Monkey^{c} | |
| Dose (mg) | Route | (μg hr/mL) | (μg/mL) | AUC^{d} | Cₘₐₓ^{e} | AUC^{d} | Cₘₐₓ^{e} |
| 420 | SC | 17 731 | 37 | 42.4 | 43 | 45.1 | 40.5 |
| (cohort 12)^{f} | | | | | | | |
| 420 | SC | 18 513.6 | 38.5 | 40.6 | 41.3 | 43.2 | 39 |
| (cohort 13)^{f} | | | | | | | |

AUC = area under the concentration-time curve; AUCₗₐₛₜ = area under the concentration-time curve during a last dosing interval; AUC₀₋₂₈ = area under the concentration-time curve during a dosing interval defined here from time 0 to 28 days after the 2nd dose; Cₘₐₓ = maximum observed drug concentration during a dosing interval defined here after the 2nd dose; FIH = First in Human; NOAEL = no observed adverse effect level; PK = pharmacokinetic; SC = subcutaneous; Q4W = every 4 weeks.
^{a}PK data from AMG 133 FIH study is used for PK prediction
^{b}After the last dose after 13 weeks of treatment (7 total doses) at the NOAEL dose of 150 mg/kg in mice (Study 150238); mean AUCₗₐₛₜ (AUC_{0-168 hrs}) of 188 000 μg hr/mL and mean Cₘₐₓ of 1590 mg/mL of Intact AMG 133.
^{c}After the last dose after 13 weeks of treatment (7 total doses) at the NOAEL dose of 120 mg/kg in cynomolgus monkeys (Study 150239); mean AUCₗₐₛₜ (AUC_{0-168 hrs}) was 200 000 μg hr/mL and mean Cₘₐₓ of 1500 mg/mL of Intact AMG 133.
^{d}AUC exposure multiples = ratio of AUCₗₐₛₜ after 13 weeks (7 doses) of AMG 133 administration in the mouse or monkey × 4/predicted AUC₀₋₂₈ in humans after the 2nd dose.
^{e}Cₘₐₓ multiple = the ratio of Cₘₐₓ after the last dose after 13 weeks (7 doses) of AMG 133 in the mouse or monkey to the predicted Cₘₐₓ for each human single dose.
^{f}Actual dose levels will be based on available data from previous cohorts.

### Source: Report 153788

TABLE 3
Estimated Exposure Margins of Total AMG 133 Following Repeat Doses (Q4W)

| | | Predicted | | Predicted Exposure | | | |
|---|---|---|---|---|---|---|---|
| | | Human Exposure | | Multiples^{a} | | | |
| Clinical | | AUCₜₐᵤ | Cₘₐₓ | Mouse^{b} | | Monkey^{c} | |
| Dose (mg) | Route | (μg hr/mL) | (μg/mL) | AUC^{d} | Cₘₐₓ^{e} | AUC^{d} | Cₘₐₓ^{e} |
| 420 | SC | 22831 | 43.5 | 51 | 54.3 | 48.9 | 45.1 |
| (cohort 12)^{f} | | | | | | | |
| 420 | SC | 24504 | 46.4 | 47.5 | 50.9 | 45.5 | 42.2 |
| (cohort 13)^{f} | | | | | | | |

AUC = area under the concentration-time curve; AUC₀₋₂₈ = area under the concentration-time curve during a dosing interval defined here from time 0 to 28 days after the 2nd dose; Cₘₐₓ = maximum observed drug concentration during a dosing interval defined here after the 2nd dose; FIH = First in Human; NOAEL = no observed adverse effect level; PK = pharmacokinetic; SC = subcutaneous; Q4W = every 4 weeks.
^{a}PK data from AMG 133 FIH study is used for PK prediction
^{b}After the last dose after 13 weeks of treatment (7 total doses) at the NOAEL dose of 150 mg/kg in mice (Study 150238); mean AUCₗₐₛₜ (AUC_{0-168 hrs}) of 291 000 μg hr/mL and mean Cₘₐₓ of 2360 mg/mL of Total AMG 133.
^{c}After the last dose after 13 weeks of treatment (7 total doses) at the NOAEL dose of 120 mg/kg in cynomolgus monkeys (Study 150239); mean AUCₗₐₛₜ (AUC_{0-168 hrs}) was 279 000 μg hr/mL and mean Cₘₐₓ of 1960 mg/mL of Total AMG 133.
^{d}AUC exposure multiples = ratio of AUCₗₐₛₜ after 13 weeks (7 doses) of AMG 133 administration in the mouse or monkey × 4/predicted (AUC₀₋₂₈ days) in humans after the 2nd dose.
^{e}Cₘₐₓ multiple = the ratio of Cₘₐₓ after the last dose after 13 weeks (7 doses) of AMG 133 in the mouse or monkey to the predicted Cₘₐₓ for each human single dose.
^{f}Actual dose levels will be based on available data from previous cohorts.

### End of Study Definition

Primary Completion: The primary completion date is defined as the date when the last subject is assessed or receives an intervention for the final collection of data for the primary endpoint(s).

If the study concludes prior to the primary completion date originally planned in the protocol (ie, early termination of the study), then the primary completion will be the date when the last subject is assessed or receives an intervention for evaluation in the study (ie, last subject last visit).

End of Study: The end of study (EOS) date is defined as the date when the last subject across all sites is assessed or receives an intervention for evaluation in the study (ie, last subject last visit), following any additional parts in the study (eg, long-term follow-up, additional antibody testing), as applicable.

### Example 3

### Phase 2 Study

### Overall Design

This is a phase 2, randomized, double-blind, dose-ranging, placebo-controlled study to evaluate the efficacy, safety, and tolerability of AMG 133 in adult subjects with overweight or obesity, with and without diabetes mellitus. Cohort A will consist of subjects without a diagnosis of type 1 or type 2 diabetes mellitus, and cohort B will consist of subjects with a diagnosis of type 2 diabetes mellitus. Potential subjects will be screened within approximately 28 days before day 1 to assess their eligibility to enroll in the study. The study will include 2 parts.

### Part 1

All subjects (cohort A and cohort B) randomized into the study will begin part 1 portion of the study.

Subjects in cohort A will be randomized on day 1 in a 3:3:3:2:2:2:3 ratio to receive AMG 133 140, 280, or 420 mg every 4 weeks (Q4W), or 420 mg every 8 weeks (Q8W) (all without dose escalation), 420 mg Q4W with 4- or 12-week dose escalation, or placebo, respectively.

Subjects in cohort B will be randomized on day 1 in a 1:1:1:1 ratio to receive AMG 133 140, 280, or 420 mg Q4W (all without dose escalation), or placebo, respectively.

Subjects will be stratified by sex. For cohort A, women will be capped at 70%. For both cohorts A and B, the study will attempt to enroll approximately 10% of subjects who had previously been treated with a glucagon-like peptide 1 (GLP-1) receptor agonist for weight management purposes and discontinued such treatment at least 90 days prior to screening.

The treatment period for part 1 will continue until week 52 with the last dose of AMG 133/placebo administered at the week 48 visit. At the week 52 visit, subjects will have the option to begin part 2 if they meet the entry criteria. Subjects who do not meet the part 2 entry criteria will proceed with the safety follow-up/end of study visit at 16 weeks (+7 days) after the last dose of AMG 133/placebo or 12 weeks (+7 days) after the week 52 visit.

### Part 2

Part 2 is optional and subjects must provide informed consent before initiation of any part 2 study-specific activities/procedures. Subjects may participate in part 2 only if they completed dosing through week 48 of part 1 and have lost at least 15% body weight from baseline (day 1) to week 52 using the following equation and using the same units for both measurements (either kg or lb):

% Reduction in body weight=([Weight measured at baseline−Weight measured at week 52]/Weight measured at baseline)×100

For part 2, subjects will be re-randomized in a double-blind manner to a new part 2 treatment group based on their treatment assignment from part 1. Subjects from group 1 will receive placebo for part 2. Subjects from group 2 will be re-randomized in a 1:1 allocation ratio to either placebo or AMG 133 70 mg Q4W. Subjects from group 3 will be re-randomized in a 2:3:3:2 ratio to receive placebo, AMG 133 140 mg Q4W, 420 mg Q4W, or AMG 133 420 mg Q12W, respectively.

For subjects who participate in part 2 of the study, treatment with AMG 133/placebo at the newly assigned part 2 dose will continue from week 52 through week 104 with a safety follow-up/end of study visit 12 weeks (+7 days) after the week 104 visit.

The primary analysis will be performed at week 52 with an administrative interim analysis planned at week 24. Administrative interim analyses may be performed throughout the study to inform on strategic considerations: however, the study team will remain blinded to treatment assignments for analyses conducted prior to the primary analysis.

### Phase 2 Treatment Summaries:

### Part 1

Subjects in the no dose-escalation treatment groups in cohort A will receive AMG 133 140, 280, or 420 mg or placebo administered subcutaneously (SC) Q4W, or AMG 133 420 mg Q8W, for 52 weeks (last dose on week 48). In addition, 2 separate dose-escalation regimens will be evaluated only in cohort A for the 420 mg dose.

Subjects in the 420 mg 4-week dose-escalation treatment group will receive AMG 133 70 mg SC on day 1 and week 2, and 420 mg Q4W from week 4 to week 48. Subjects in the 420 mg 12-week dose-escalation treatment group will receive 70 mg SC on day 1, 140 mg on week 4, 280 mg on week 8, and 420 mg Q4W from week 12 to week 48. To ensure blinding, subjects in cohort A assigned to AMG 133 treatment groups who, by design, are not to receive AMG 133 at an administration visit in the schedule of activities (Section 1.3) will receive placebo at those visits. Subjects in cohort B will receive AMG 133 140, 280, or 420 mg or placebo administered SC Q4W for 52 weeks (last dose on week 48).

### Part 2

Subjects in group 1 will receive placebo administered SC Q4W, subjects in group 2 will receive either placebo or AMG 133 70 mg administered SC Q4W. and subjects in group 3 will receive either placebo administered SC Q4W AMG 133 140 or 420 mg administered SC Q4W, or AMG 133 420 mg administered SC Q12W.

We claim:

1. A method for treating obesity in a patient in need thereof comprising administering to the patient a pharmaceutical composition comprising an antagonistic anti-GIPR antibody conjugated to a GLP-1R agonist at a dose of about 21 mg to about 840 mg every four weeks, a dose of about 21 mg to about 840 mg every six weeks, a dose of about 21 mg to about 840 mg every eight weeks, or a dose of about 21 mg to about 840 mg every twelve weeks.

2. The method of claim 1, wherein the dose is about 21 mg, about 70 mg, about 140 mg, about 280 mg, about 420 mg, about 560 mg, or about 840 mg every four weeks; about 21 mg, about 70 mg, about 140 mg, about 280 mg, about 420 mg, about 560 mg, or about 840 mg every six weeks; about 21 mg, about 70 mg, about 140 mg, about 280 mg, about 420 mg, about 560 mg, or about 840 mg every eight weeks, or about 21 mg, about 70 mg, about 140 mg, about 280 mg, about 420 mg, about 560 mg, or about 840 mg every twelve weeks.

3. The method of claim 1, wherein the dose is about 280 mg every four weeks; about 280 mg every six weeks, about 280 mg every eight weeks, or about 280 mg every twelve weeks.

4. The method of claim 1, wherein the dose is about 280 mg every four weeks.

5. The method of claim 1, wherein the dose is about 420 mg every four weeks; about 420 mg every six weeks, about 420 mg every eight weeks, or about 420 mg every twelve weeks.

6. The method of claim 1, wherein the dose is about 420 mg every four weeks.

7. The method of claim 1, wherein the pharmaceutical composition is administered once every four weeks.

8. The method of any one of claim 1 wherein the pharmaceutical composition is administered parenterally.

9. The method of claim 8, wherein the parenteral administration is subcutaneous administration.

10. The method of claim 1, wherein the pharmaceutical composition is administered to the patient with a syringe pre-filled with the pharmaceutical composition.

11. The method of claim 1, wherein the pharmaceutical composition is administered to the patient with an autoinjector.

12. The method of claim 1, wherein administration of the pharmaceutical composition does not substantially cause an adverse side effect in the patient.

13. The method of claim 1, wherein the patient has a body mass index (“BMI”) greater than 30 kg/m2, 35 kg/m2, or 40 kg/m2.

14. The method of claim 1, wherein the patient has or is diagnosed with diabetes.

15. The method of claim 1, wherein the patient has not previously received therapy for obesity.

16. The method of claim 1, wherein the anti-GIPR antibody comprises a CDRH1 comprising the amino acid sequence of SEQ ID NO:5, a CDRH2 comprising the amino acid sequence of SEQ ID NO:6, a CDRH3 comprising the amino acid sequence of SEQ ID NO:7, a CDRL1 comprising the amino acid sequence of SEQ ID NO:8, a CDRL2 comprising the amino acid sequence of SEQ ID NO:9, and a CDRL3 comprising the amino acid sequence of SEQ ID NO:10.

17. The method of claim 1, wherein the antagonistic anti-GIPR antibody comprises a heavy chain comprising the amino acid sequence of SEQ ID NO:1, and a light chain comprising the amino acid sequence of SEQ ID NO:2.

18. The method of claim 1, wherein the GLP-1R agonist comprises the amino acid sequence of SEQ ID NO:3.

19. The method of claim 1, wherein the antagonistic anti-GIPR antibody is linked to the GLP-1R agonist via a peptide linker comprising SEQ ID NO: 4.

20. The method of claim 1, wherein the pharmaceutical composition further comprises a buffer.

21. The method of claim 20, wherein the buffer is an acetate buffer.

22. The method of claim 1, wherein the pharmaceutical composition further comprises a surfactant.

23. The method of claim 22, wherein the surfactant is polysorbate 20 or polysorbate 80.

24. The method of claim 1, wherein the pharmaceutical composition further comprises a stabilizing agent.

25. The method of claim 24, wherein the stabilizing agent is sucrose.

26-49. (canceled)

50. A method for treating obesity in a patient in need thereof comprising administering to the patient a pharmaceutical composition comprising an antagonistic anti-GIPR antibody conjugated to a GLP-1R agonist at a dose of about 21 mg, about 70 mg, about 140 mg, about 280 mg, about 420 mg, about 560 mg, or about 840 mg every four weeks; about 21 mg, about 70 mg, about 140 mg, about 280 mg, about 420 mg, about 560 mg, or about 840 mg every six weeks; about 21 mg, about 70 mg, about 140 mg, about 280 mg, about 420 mg, about 560 mg, or about 840 mg every eight weeks, or about 21 mg, about 70 mg, about 140 mg, about 280 mg, about 420 mg, about 560 mg, or about 840 mg every twelve weeks,
wherein
the anti-GIPR antibody comprises a CDRH1 comprising the amino acid sequence of SEQ ID NO:5, a CDRH2 comprising the amino acid sequence of SEQ ID NO:6, a CDRH3 comprising the amino acid sequence of SEQ ID NO:7, a CDRL1 comprising the amino acid sequence of SEQ ID NO:8, a CDRL2 comprising the amino acid sequence of SEQ ID NO:9, and a CDRL3 comprising the amino acid sequence of SEQ ID NO:10; and
the GLP-1R agonist comprises the amino acid sequence of SEQ ID NO:3.

51. The method of claim 50, wherein the dose is about 280 mg every four weeks; about 280 mg every six weeks, about 280 mg every eight weeks, or about 280 mg every twelve weeks.

52. The method of claim 50, wherein the dose is about 280 mg every four weeks.

53. The method of claim 50, wherein the dose is about 420 mg every four weeks; about 420 mg every six weeks, about 420 mg every eight weeks, or about 420 mg every twelve weeks.

54. The method of claim 50, wherein the dose is about 420 mg every four weeks.

55. The method of claim 50, wherein the pharmaceutical composition is administered once every four weeks.

56. The method of claim 50, wherein the pharmaceutical composition is administered parenterally.

57. The method of claim 57, wherein the parenteral administration is subcutaneous administration.

58. The method of claim 50, wherein the pharmaceutical composition is administered to the patient with a syringe pre-filled with the pharmaceutical composition.

59. The method of claim 50, wherein the pharmaceutical composition is administered to the patient with an autoinjector.

60. The method of claim 50, wherein administration of the pharmaceutical composition does not substantially cause an adverse side effect in the patient.

61. The method of claim 50, wherein the patient has a body mass index (“BMI”) greater than 30 kg/m2, 35 kg/m2, or 40 kg/m2.

62. The method of claim 50, wherein the patient has or is diagnosed with diabetes.

63. The method of claim 50, wherein the patient has not previously received therapy for obesity.

64. The method of claim 50, wherein the antagonistic anti-GIPR antibody comprises a heavy chain comprising the amino acid sequence of SEQ ID NO:1, and a light chain comprising the amino acid sequence of SEQ ID NO:2.

65. The method of claim 50, wherein the antagonistic anti-GIPR antibody is linked to the GLP-1R agonist via a peptide linker comprising SEQ ID NO: 4.

66. The method of claim 50, wherein the pharmaceutical composition further comprises a buffer.

67. The method of claim 66, wherein the buffer is an acetate buffer.

68. The method of claim 50, wherein the pharmaceutical composition further comprises a surfactant.

69. The method of claim 68, wherein the surfactant is polysorbate 20 or polysorbate 80.

70. The method of claim 50, wherein the pharmaceutical composition further comprises a stabilizing agent.

71. The method of claim 70, wherein the stabilizing agent is sucrose.

72-92. (canceled)
